SEQUENCE LISTING (14 sequences)

SEQ ID NO: 1 - protein, 108 aa, Artificial Sequence
  Description of Artificial Sequence Synthetic polypeptide
DIQMTQSPSSLSASVGDRVTITCRASQGISSNLNWYQQKPGKAPKLLIYAASNLQSGPSRFSGSGSGTDFTLTISSIQPEDFAVYYCGGYSDQSYTFGQGTKVEIKRT

SEQ ID NO: 2 - protein, 126 aa, Artificial Sequence
  Description of Artificial Sequence Synthetic polypeptide
QVQLVESGGGLVQPGGSLRLSCAASGFTFSSYGMSWVRQAPGKGLEWVSSISYSDSNTYYADSVKGRFTISRDNSKNTLYLGMNSLRAEDTAVYYCARGLGDYGHHHGLSGIFDYWGGGTLVTVSS

SEQ ID NO: 3 - protein, 109 aa, Artificial Sequence
  Description of Artificial Sequence Synthetic polypeptide
DIELTQPPSVSVAPGGTARISCSGDSIGEQYAHWYQQKPGQAPVLVIYDDNKRPSGIPERFSGSNSGNTATLTISGTQAEDEADYYCGSYTLTNTASVFGGGTKLTVLG

SEQ ID NO: 4 - protein, 125 aa, Artificial Sequence
  Description of Artificial Sequence Synthetic polypeptide
QVQLVESGGGLVQPGGSLRLSCAASGFTFSNYAMNWVRQAPGKGLEWVSRISYSGSDTYYADSVKGRFTISRDNSKNTLYLGRNNSLRAEDTAVYYCAREGEFGFMYSTLVFDSWGQGTLVTVSS

SEQ ID NO: 5 - protein, 109 aa, Artificial Sequence
  Description of Artificial Sequence Synthetic polypeptide
DIELTQPPSVSVAPGQTARISCSGDSIGEQYAHWYQQKPGQAPVLVIYDDNKRPSGIPERFSGSNSGNTATLTISGTQAEDEADYYCSSYTYSSDASVFGGGTKLTVLG

SEQ ID NO: 6 - protein, 123 aa, Artificial Sequence
  Description of Artificial Sequence Synthetic polypeptide
QVQLVESGGGLVQPGGSLRLSCAASGFTFSNYAMNWVRQAPGKGLEWVSAIHDNGHTYYPDSVKGRFTISRDNSKNTLYLQMNSLRAEDTAVYYCAREGEFGFMYSTLVFDSWGGGTLVTVSS

SEQ ID NO: 7 - protein, 108 aa, Artificial Sequence
  Description of Artificial Sequence Synthetic polypeptide
DIQMTGSPSSLSASVGDRVTITCRASQGISSNLNWYQQKPGKAPKLLIYAASNLQSGPSRFSGSGSGTDFTLTISSLQPEDFATYYCQQYASPRQTFGQGTKVEIKRT

SEQ ID NO: 8 - protein, 128 aa, Artificial Sequence
  Description of Artificial Sequence Synthetic polypeptide
QVQLVESGGGLVQPGGSLRLSCAASGFTFSSYGMSWVRQAPGKGLEWVSGIRAKQSGYATDYAAPVKGRFTISRDNSKNTLYLGMNSLRAEDTAVYYCARGLGDYGHHHGLSGIFDYWGQGTLVTVSS

SEQ ID NO: 9 - protein, 108 aa, Artificial Sequence
  Description of Artificial Sequence Synthetic polypeptide
DIQMTQSPSSLSASVGDNVTITCRASQGISSNLNWYQQKPGKAPKLLIYAASNLQSGPSRFSGSGSGTDFTLTISSLQPEDFATYYCQQYEFGIQTFGQGTKVEIKRT

SEQ ID NO: 10 - protein, 128 aa, Artificial Sequence; identical to SEQ ID NO: 8
  Description of Artificial Sequence Synthetic polypeptide

SEQ ID NO: 11 - protein, 108 aa, Artificial Sequence
  Description of Artificial Sequence Synthetic polypeptide
DIQMTQSPSSLSASVGDRVTITCRASQGISSNLNWYQQKPGKAPKLLIYAASNLQSGPSRFSGSGSGTDFTLTISSLQPEDFATYYCQQYSSNPQTFGQGTKVEIKRT

SEQ ID NO: 12 - protein, 128 aa, Artificial Sequence
  Description of Artificial Sequence Synthetic polypeptide
QVQLVESGGGLVQPGGSLRLSCAASGFTFSSYGMSWVRQAPGKGLEWVSFIEPKWRGGATHYAASVKGRFTISRDNSKNTLYLQMNSLRAEDTAVYYCARGLGDYGHHHGLSGIFDYWGQGTLVTVSS

SEQ ID NO: 13 - protein, 108 aa, Artificial Sequence
  Description of Artificial Sequence Synthetic polypeptide
DIQMTQSPSSLSASVGDRVTITCRASQGISSNLNWYQQKPGKAPKLLIYAASNLQSGPSRFSGSGSGTDFTLTISSLQPEDFAVYYCQQYSDQSYTFGQGTKVEIKRT

SEQ ID NO: 14 - protein, 128 aa, Artificial Sequence
  Description of Artificial Sequence Synthetic polypeptide
QVQLVESGGGLVQPGGSLRLSCAASGFTFSSYGMSWVRQAPGKGLEWVSGIRAKQSGYATDYAAPVKGRFTISRDNSKNTLYLQMNSLRAEDTAVYYCARGLGDYGHHHGLSGIFDYWGQGTLVTVSS

(19) United States
(12) Patent Application Publication
(10) Pub. No.: US 2009/0081207 A1
(43) Pub. Date: Mar. 26, 2009

(54) High affinity human and humanized anti-alpha5beta1 integrin function blocking antibodies with reduced immunogenicity

(76) Inventors: ANDREAS MENRAD, ELY CAMBS (GB); JOERG WILLUDA, BERLIN (DE); KLAUS BOSSLET, BERLIN (DE); DIETER ZOPF, BERLIN (DE); HEIKE PETRUL, BERLIN (DE); STEFAN STEIDL, MUNCHEN (DE); JOSEF PRASSLER, GERMERING (DE); CORINNE PETIT-FRERE, MUNCHEN (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/802,573

(22) Filed: May 23, 2007

Related U.S. Application Data
(60) Provisional application No. 60/811,096, filed on Jun. 6, 2006.

(30) Foreign Application Priority Data
May 24, 2006 (EP) 06010779.4

(51) Int. Cl. A61K 39/395; C07K 16/18; C12N 9/96; C12N 15/11; C12N 15/00; A61P 31/00; G01N 33/574; C12N 5/06; A01K 67/027; C12P 21/04

(52) U.S. Cl. 424/133.1; 530/387.3; 530/387.1; 530/391.7; 530/391.3; 435/188; 536/23.53; 435/320.1; 435/325; 800/13; 800/6; 435/69.6; 435/7.23

(57) ABSTRACT

The present invention relates to recombinant human or humanized polypeptides which bind to α5β1 integrin with high affinity and blocking function. Further, diagnostic and pharmaceutic applications of the potypeptides are disclosed.

The present invention relates to recombinant human or humanized polypeptides which bind to α5β1 integrin with high affinity and blocking function. Further, diagnostic and pharmaceutic applications of the polypeptides are disclosed.

Angiogenesis is the process by which new blood vessels develop from pre-existing vessels. The growth of new blood vessels promotes embryonic development, wound healing, and the female reproductive cycle. It also plays an important role in the pathological development of solid cancers and other diseases e.g. haemangiomas, diabetic retinopathy, age-related macular degeneration, psoriasis, rheumatoid arthritis and possibly osteoarthritis and inflammatory bowel disease (1).

Growth factors released by hypoxic tumor tissue stimulate the growth of new blood vessels. While growth factors and their receptors play key roles in angiogenic sprouting, adhesion to the extracellular matrix (ECM) also is a prime-regulator of angiogenesis. Adhesion promotes endothelial cell survival, as well as endothelial proliferation and migration (2-5). One ECM protein in particular, fibronectin, is expressed in provisional (tumor) matrices and provides proliferative signals to vascular cells (2,3). Notably, fibronectin-null mice die early in development from a collection of defects, which include an improperly formed vasculature (6,7).

Studies in experimental animal models and in mutant mice indicate that the α5β1 integrin which is the most important receptor for fibronectin plays a key role in regulating angiogenesis. Embryonic deletion of this integrin induces early and lethal mesenchymal abnormalities, which include defects in the organisation of the emerging vasculature (8,9) and defects in the ability of endothelial cells to form vessel-like structures in vitro (10,11).

The expression of the α5β1 integrin is specifically associated with angiogenesis: it is not detectable in quiescent endothelium but expressed in response to angiogenic growth factors (3,4) in vitro or within the angiogenic vasculature of a growing tumor in vivo (12, 20, 21).

Kim et al. (3) could demonstrate that the mouse anti-α5β1 integrin-function blocking antibody IIA1 inhibits both growth factor-induced and tumor angiogenesis in vivo. Studies of the signals transduced when this integrin is antagonised indicate that the unligated receptor activates PKA, which then activates caspase 3 and 8 and induces apoptosis (2,13).

Attempts have been carried out to prepare humanized derivatives of the mouse antibody IIA1 (BD Pharmingen Cat. No. 555614). As a result, a 82% human/18% mouse chimeric IgG4 monoclonal antibody termed M200, was generated. Further, a monovalent Fab-fragment of M200, temed F200, has been generated and successfully tested in a cynomoigus monkey model for macular degeneration. Further attempts to prepare fully humanized antibody derivatives of M200, however, resulted in a dramatic loss of bioactivity (14).

Any application of presently known antibodies against α5β1 integrin such as M200 or F200 in human medicine has the risk of inducing an immunogenic human anti-chimeric antibody (HACA) response in human patients. Thus, an object of the present invention was to provide human anti-α5β1 integrin antibodies which have reduced immunogenicity compared to existing chimeric antibodies while retaining target-specificity and high bioactivity and affinity.

According to the present invention, fully human antibodies in the Fab format were isolated from a HuCAL®-Gold antibody library by phage display using α5β1 integrin transfected cells. These antibodies show high in vitro activity while low immunogenicity can be expected in human patients due to the fully human origin.

Thus, a first aspect of the present invention is a human or humanized antibody or an antigen-binding fragment thereof which (i) binds to α5β1 integrin with an affinity of 100 nM and preferably ≦10 nM and (ii) inhibits the adhesion of α5β1 integrin expressing cells to its receptor in vitro and in vivo.

The polypeptide of the present invention is a human or humanized antibody or an antigen-binding fragment thereof. The term “human antibody” according to the present invention relates to antibody molecules which have substantially human or fully human variable domains and, if present, human constant domains. The term “human” as used in the present application relates to sequences which can be formed in individual human beings or by use of consensus sequences resulting therefrom, e.g. as described in the corresponding compendium by Kabat et al. (1991), Sequences of Proteins of immunological Interest, 5^{th} Edition, NIH Publication no. 91-3242, US Department of Health and Human Services, Washington, D.C., which is herein incorporated by reference. The term “substantially human” refers to sequences which may differ from “fully human” sequences as described by Kabat et al. in up to 1, 2, 3, 4 or 5 amino acids. More particularly, the antibodies or antibody fragments according to the present invention comprise substantially or fully human variable framework regions in the heavy (H) and light (L) immunoglobulin chains. The term “humanized antibody” in the sense of the present invention relates to antibody molecules which have substantially murine or fully murine variable domains and human or substantially human constant domains, and which are >82%, preferably at least 90%, and especially preferably at least 98% human. The term “murine” as used in the present application relates to sequences which can be formed in individual rodents or by use of consensus sequences resulting therefrom. The term “substantially murine” refers to sequences which may differ from “fully murine” sequences in up to 1, 2, 3, 4 or 5 amino acids. Preferably, the antibody or antibody fragment thereof is an IgG antibody, e.g. a human or humanized IgG1, IgG2, IgG3 or IgG4 antibody or a fragment thereof, e.g. a Fab, Fab′ or (Fab)₂ fragment. The present invention, however, also relates to recombinant antibodies having human sequences, e.g. single chain (sc) antibodies or fragment thereof, e.g. scFv fragments.

The antibodies or antibody fragments of the present invention contain one or more antigen-binding sites which specifically interact with α5β1 integrin. Preferably, this antigen-binding properties are obtained by combining a variable heavy chain (VH) and a variable light chain (VL) region. A VH or VL region includes framework regions (FR1, FR2, FR3, and FR4) and antigen binding-mediating CDR regions (H-CDR1, H-CDR2, H-CDR3 for the VH region and L-CDR1, L-CDR2, L-CDR3 for the VL region).

The human or humanized antibody or antibody fragment of the invention preferably has an affinity for the α5β1 integrin corresponding to a K_{D} value of ≦100 nM, preferably ≦10 nM and most preferably ≦1 nM, wherein the affinity is determined by FACS-titration on α5β1 positive human HUVEC cells as described in the Examples or by competition BIAcore or competition ELISA measurement.

Further, the polypeptides of the invention inhibit the adhesion of an α5β1 integrin expressing human tumor cell as described in the Examples, for example the K562 cell (ATCC accession number: CCL-243) studied by Lozzio et al. (1979), Leukemia Research, 3: 363-370, in vitro. Preferably, the antibody or antibody fragment shows a 50% inhibition of cell adhesion at a concentration (IC50) of ≦10 nM and preferably ≦5 nM.

Further, the polypeptides of the invention preferably are capable of inducing caspase activity in HUVEC cells. The IC50 value with regard to HUVEC viability is preferably ≦10 nM, more preferably ≦5 nM, wherein the IC50 value (50% viability) is determined as described in the Examples.

Further, the polypeptides, antibodies and antibody fragments of the invention can preferably be used for diagnosis and for prevention and treatment of tumors and cancer, especially colon carcinoma.

Said polypeptides, antibodies and antibody fragments can be conjugated with detectable labelling groups, such as radioactive, NMR, dye, enzyme and fluorescent labelling groups. Radioactive groups can be, for example I¹²⁵, I¹³¹ or Y⁹⁰.

Preferably, the antibody or antibody fragment of the invention comprises:

(a) a VH region selected from
(i) amino acid sequence SEQ ID NO: 1 (MOR04624), SEQ ID NO: 3 (MOR04055) or at least one H-CDR1, H-CDR2 and/or H-CDR3 region of one of said VH regions, or
(ii) an amino acid sequence derived from a sequence of (i) by alteration of at least one H-CDR region, and/or
   (b) a VL region selected from
(i)
amino acid sequence SEQ ID NO: 2 (MOR04624), SEQ ID NO: 4 (MOR04055) or at least one L-CDR1, L-CDR2 and/or L-CDR3 region of one of said VL regions, or
(ii)
an amino acid sequence derived from a sequence of (i) by alteration of at least one L-CDR region.

Especially preferred is an antibody or antibody fragment comprising a VH region derived from a VH-region of (a) (i) as described above by randomization of the H-CDR2 region.

In another especially preferred embodiment the antibody or antibody fragment comprises a VL-region derived from a VL-region of (b) (i) as described above by randomization of the L-CDR3 region.

In still another especially preferred embodiment the antibody or antibody fragment comprises a VH- and/or a VL-region derived from a VH-region of (a) (i) and/or a VL-region of (b) (i) by shuffling of the antibody chains.

Sublibraries of H-CDR2 and L-CDR3 are generated by exchange of H-CDR2 and L-CDR3, respectively, with human CDR repertoires by methods of protein engineering (17).

For example the antibody or antibody fragment comprises a VH and/or VL region derived from the VL and/or VH region as described in SEQ ID NO: 1 or SEQ ID NO: 2 (MOR04624). Especially preferred is a polypeptide comprising:
(a) a VH-region selected from amino acid sequence SEQ ID NO: 5 (MOR04971), SEQ ID NO: 7 (MOR04974), SEQ ID NO: 9 (MOR04975), SEQ ID NO: 11 (MOR04977), and SEQ ID NO. 11 (MOR04985) or at least one H-CDR1, H-CDR2 and/or H-CDR3 region of said VH-regions, and/or
(b) a VL-region selected from amino acid sequence SEQ ID NO: 6 (MOR04971), SEQ ID NO: 8 (MOR04974), SEQ ID NO: 10 (MOR04975), SEQ ID NO: 12 (MOR04977) and SEQ ID NO: 14 (MOR04985), or at least one L-CDR1, L-CDR2 and/or L-CDR3 region of said VL-region.

Specific examples of polypeptides of the present invention are as follows:

An antibody or antibody fragment comprising the VH region of SEQ ID NO: 1 and the VL region of SEQ ID NO: 2 (MOR04624) or at least one H-CDR1-, H-CDR-2, H-CDR3-, L-CDR1-, L-CDR2- or L-CDR3-region thereof.

An antibody or antibody fragment comprising the VH region of SEQ ID NO: 3 and the VL region of SEQ ID NO: 4 (MOR04055) or at least one H-CDR1-, H-CDR-2, H-CDR3-, L-CDR1-, L-CDR2- or L-CDR3-region thereof.

An antibody or antibody fragment comprising the VH region of SEQ ID NO: 5 and the VL region of SEQ ID NO: 6 (MOR04971) or at least one H-CDR1-, H-CDR-2, H-CDR3-, L-CDR1-, L-CDR2- or L-CDR3-region thereof.

An antibody or antibody fragment comprising the VH region of SEQ ID NO: 7 and the VL region of SEQ ID NO: 8 (MOR04974) or at least one H-CDR1-, H-CDR-2, H-CDR3-, L-CDR1-, L-CDR2- or L-CDR3-region thereof.

An antibody or antibody fragment comprising the VH region of SEQ ID NO: 9 and the VL region of SEQ ID NO: 10 (MOR04975) or at least one H-CDR1-, H-CDR-2, H-CDR3-, L-CDR1-, L-CDR2- or L-CDR3-region thereof.

An antibody or antibody fragment comprising the VH region of SEQ ID NO: 11 and the VL region of SEQ ID NO: 12 (MOR04977) or at least one H-CDR1-, H-CDR-2, H-CDR3-, L-CDR1-, L-CDR2- or L-CDR3-region thereof.

An antibody or antibody fragment comprising the VH region of SEQ ID NO: 13 and the VL region of SEQ ID NO: 14 (MOR04985) or at least one H-CDR1-, H-CDR-2, H-CDR3-, L-CDR1-, L-CDR2- or L-CDR3-region thereof.

The invention also refers to antibodies or antibody fragments which are directed against the same epitope on the antigen as the above-mentioned preferred and/or exemplified antibodies or antibody fragments.

The VH and VL chain of the polypeptide comprises the following regions:

VH chain of MOR04624, MOR04055 and derivatives (numbering scheme according to (17)):

Framework 1 region extends from amino acid 1 to 30aa

CDR1 region extends from amino acid 31 to 35 aa

Framework 2 region extends from amino acid 36 to 49aa

CDR2 region extends from amino acid 50 to 65aa

Framework 3 region extends from amino acid 66 to 94aa

CDR3 region extends from amino acid 95 to 102aa

Framework 4 region extends from amino acid 103 to 113aa

VLκ1 chain of MOR04624 and derivatives (numbering scheme according to (17)):

Framework 1 region extends from amino acid 1 to 23aa

CDR1 region extends from amino acid 24 to 35aa

Framework 2 region extends from amino acid 36 to 50aa

CDR2 region extends from amino acid 51 to 57aa

Framework 3 region extends from amino acid 59 to 89aa

CDR3 region extends from amino acid 90 to 98aa

Framework4 region extends from amino acid 99 to 109aa

VLκ1 chain of MOR04055 and derivatives (numbering scheme according to (17)):

Framework 1 region extends from amino acid 1 to 23aa

CDR1 region extends from amino acid 24 to 35aa

Framework 2 region extends from amino acid 36 to 50aa

CDR2 region extends from amino acid 51 to 57aa

Framework 3 region extends from amino acid 58 to 89aa

CDR3 region extends from amino acid 90 to 98aa

Framework 4 region extends from amino acid 99 to 109aa

The framework regions of the VH- and/or VL-chain may be altered by exchange of one or more amino acids, e.g. 1, 2, 3, 4 or 5 amino acids. For example, the framework 3 region of the VLκ1 chain may be altered in members of the MOR04624 family. Preferably, the amino acid at position 85 of the Fab sequence is exchangeable, with an exchange of valine (MOR04624, MOR04985) to threonine (MOR04974, -75, -77) being especially preferred. Further, the framework 1 region of the VH chain may be altered. In a preferred embodiment, the amino acid at position 3 of each VH-Fab sequence may be exchanged. Especially preferred is an exchange of glutamine (q) to glutamic acid (e) which may, for example, occur during cloning.

The polypeptide of the invention is suitable for therapeutic or diagnostic applications, e.g. for in vitro or in vivo diagnostic applications.

For therapeutic applications, the antibody or antibody fragment may be used as such. Alternatively, the polypeptide may be in the form of a conjugate with a therapeutic agent, for example selected from radiotherapeutical agents or chemotherapeutical agents, e.g. low molecular weight or biologic cytostatic or cytotoxic agents. The therapeutic agent may be conjugated to the antibody or antibody fragment according to known methods, preferably via a covalent linkage to reactive amino, carboxy, hydroxy and/or sulphhydryl groups of the polypeptide, optionally using homo- or hetero-bifunctional linkers.

In a further embodiment, the polypeptide may be in the form of a fusion protein comprising an antibody or antibody fragment domain and a heterologous fusion domain, e.g. a cytokine such as IL-2, IL-12 or TNF-α. Other therapeutically relevant fusion partners of the antibodies or antibody fragments according to the invention comprise engineered IgG Fc-parts for increased or decreased immunoeffector cell recruitment, protein toxins such as RNAses or ETA, small drug molecules such as maytansine or auristatin derivatives, enzymes for prodrug activation, fusion proteins with other integrin function blocking antagonists, or fusion proteins with enzymes having antiangiogenic activity such as MMP-2 or MMP-9 (15). Further, the fusion protein may be in the form of a bispecific antibody which comprises at least one α5β1 integrin binding domain as described above and a binding domain specific for a further antigen. For example, the second antigen binding domain may be directed against chelating agents for diagnostic and/or therapeutic radionucleotides, e.g. alpha, beta or gamma emitting radionuclides such as ⁹⁰Y, diagnostic NIR (near-infrared) dyes, therapeutically active dyes, surface molecules on immunological effector cells, e.g. NK-cells, cytotoxic T-cells or NK T-cells, functional blocking anti-VEGF binding domains and function blocking binding domains against VEGF receptor 1, 2 and 3 and cytokines such as interleukins.

For diagnostic applications, the polypeptide may be in the form of a conjugate with a detectable labelling group, e.g. a labelling group for an in vitro or in vivo diagnostic application. For example, the detectable labelling group may be selected from radioactive, NMR, dye, enzyme and fluorescent (e.g. NIR fluorescent) labelling groups.

For therapeutic applications, the polypeptide is preferably formulated into a pharmaceutical composition which may additionally comprise further active ingredients and/or pharmaceutically acceptable carriers, diluents and/or adjuvants. The pharmaceutical composition comprises the active agent in a therapeutically active dose, which may be determined by a skilled person according to standard methods, e.g. by in vitro experiments or in animal models. The composition is preferably administered by infusion, injection or inhalation. The dose of the active ingredient is determined according to the type and the severity of the disorder and the constitution of the patient to be treated. Preferably, the therapeutic composition is administered in several doses over a time of at least 2-4 weeks. In this context, it is referred to known protocols for the administration of antibodies or antibody conjugates, e.g. as described in Ferrara et al. Nature Reviews Drug Discovery, Vol. 3, May 2004, 391-400 and Salgaller, Current Opinion in Molecular Therapeutics, 2003, 5(6), 657-667 or to protocols for administering pharmaceutical antibodies like Rituximab, Campath, Remicade etc.

Further, the invention relates to a diagnostic composition comprising an antibody or antibody fragment as described above as a diagnostic reagent. The diagnosic composition may comprise further diagnostically acceptable reagents, carriers, diluents and/or adjuvants. The diagnostic composition comprises the polypeptide in an amount sufficient to allow diagnostic detection in the respective assay format, e.g. in an in vivo or in vitro diagnostic assay format.

The composition may be used for therapeutic or diagnostic applications in α5β1 integrin associated disorders. For example, these disorders may be hyperproliferative disorders, e.g. disorders associated with angiogenesis and/or metastasis, particularly cancer. Cancers which may be treated by the composition according to the invention particularly comprise all kinds of solid tumors, e.g. cancers of the colon, kidney, lung, prostate, breast, brain, stomach, liver or skin. Alternatively, the compositions may be employed in the treatment of hematological cancers associated with angiogenesis. Other disorders associated with neovascularization comprise, but are not limited to, endometriosis, hemangioma, rheumatoid arthritis, osteoarthritis, artheriosclerotic plaques, inflammatory bowel disease, inflammatory CNS disease, Psoriasis, eye disorders such as diabetic retinopathy or age-related macular disease, and hypertrophic scars. In a preferred embodiment the antiangiogenic activity of the composition is independent of growth factors.

The composition may comprise one or several antibodies or antibody fragments, e.g. a combination of antibodies or antibody fragments binding to different domains of α5β1 integrin. The composition may also contain small molecule drugs for combination therapy. The composition is suitable for application in human and veterinary medicine. Especially preferred is an application in human medicine.

Further, the present invention relates to a nucleic acid encoding an antibody or antibody fragment or fusion polypeptide as described above. The nucleic acid may be e.g. a single stranded or double stranded DNA or RNA. Preferably, the nucleic acid is operatively linked to an expression control sequence, which allows expression in a suitable host cell or host organism. The nucleic acid may be present on a vector or a vector system, (i.e. a plurality of vectors) which may be introduced into a host cell or host organism. The vector may be a prokaryotic vector suitable for prokaryotic cells, e.g. a plasmid or bacteriophage. Further, the vector may be an eukaryotic vector for eukaryotic host cells or host organisms, e.g. a plasmid, an artificial chromosome or a viral vector. Suitable vectors are described e.g. in Sambrook et al. (1989), Molecular Cloning, a Laboratory Manual, Cold Spring Harbor Laboratory Press and Ausubel et al. (1989), Current Protocols in Molecular Biology, John Wiley and Sons.

The present invention also refers to a cell, e.g. a prokaryotic cell or an eukaryotic cell such as a human cell which is transformed with a nucleic acid or a vector as described above. Furthermore, the invention relates to a non-human organism, e.g. a transgenic animal, such as a transgenic non-human mammal, which is transformed with a nucleic acid or vector as described above. The term “transformation” includes all methods for introducing foreign nucleic acids into a cell or an organism including transfection or infection.

The polypeptide may be prepared by cultivating a cell or a non-human organism as described above under conditions under which the polypeptide are expressed and the expressed polypeptide is recovered, e.g. from the cell, culture medium, organism or excretion products of the organism.

Further, the present invention shall be explained in detail in the following Figures and Examples:

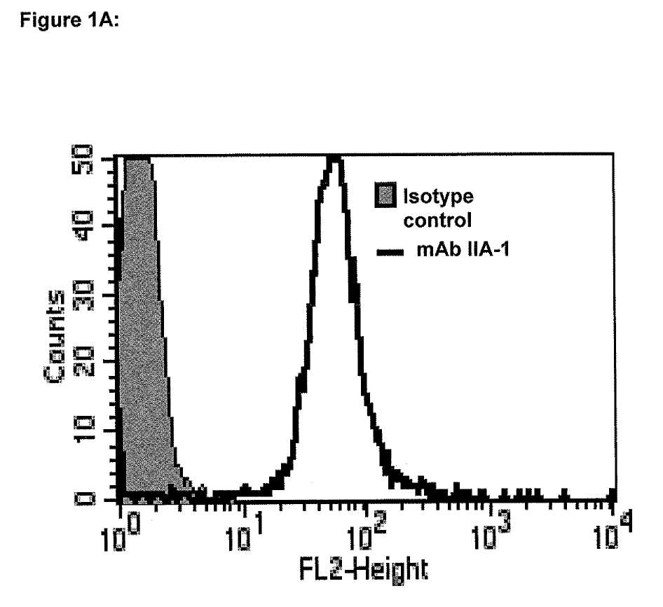

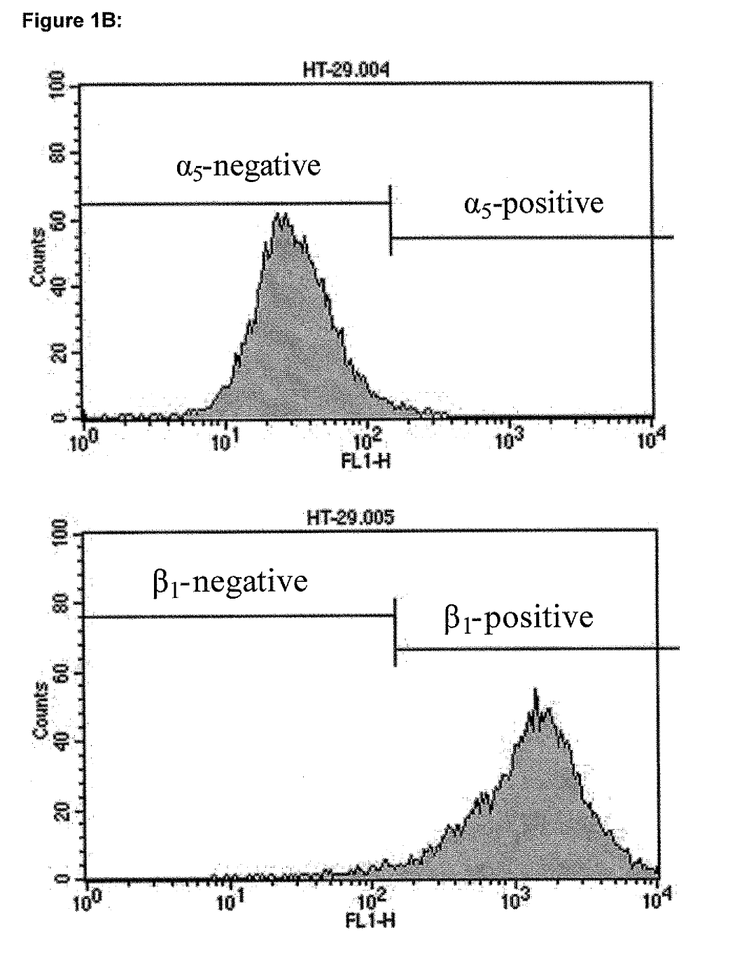

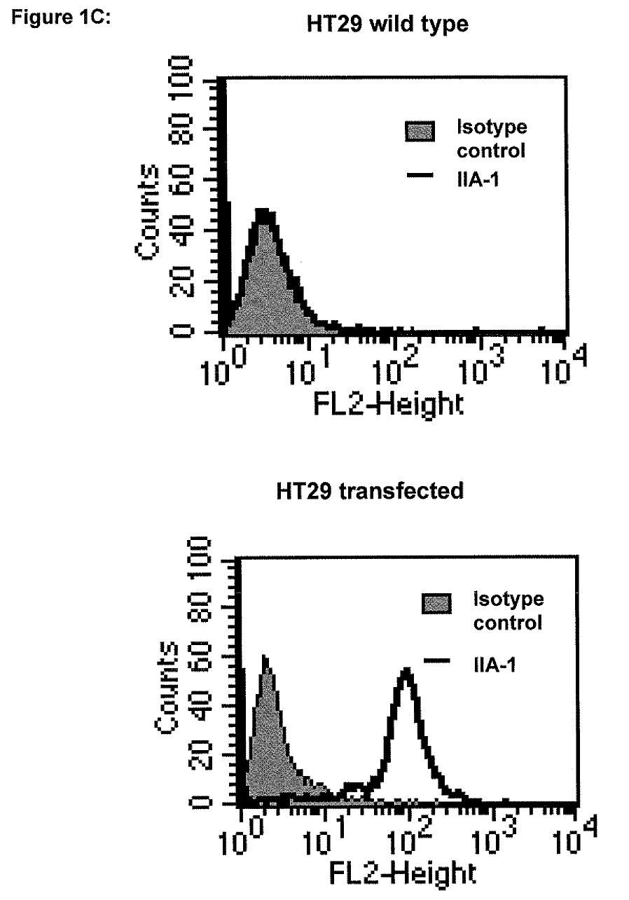

FIG. 1A: FACS Analysis of K562 cells for α5 expression: The expression of the human α5β1 integrin on the cell surface of living K562-cells was demonstrated with the function-blocking anti α5β1 integrin mouse monoclonal antibody IIA1 (14). For this purpose, standard FACS-procedures were used as described in the HuCAL® GOLD Manual provided by MorphoSys.

FIG. 1B: The human colon carcinoma cell line HT29 does not express the α5-integrin chain.

FACS analysis demonstrated that HT29 cells do not express the α5-integrin chain, whereas the β1 chain is present on the cell surface at a high density, For this reason, HT29 cells are excellently suited for the transfection with the α5-integrin chain.

FIG. 1C: The human colon carcinoma cell line HT29 expresses the α5β1 integrin after transfection with the α5-integrin cDNA. After transfection with the α5-integrin chain, the homogenous expression of the α5β1 integrin on the surface of the HT29α5 cells was demonstrated by FACS analysis using the function-blocking mouse anti human α5β1-integrin monoclonal antibody IIA1 as the reference.

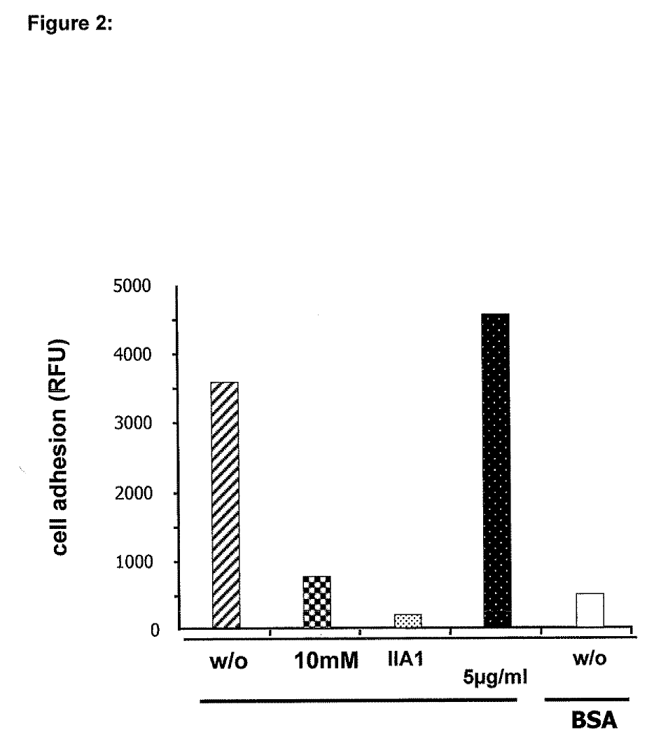

FIG. 2: Inhibition of the adhesion of K562 cells to fibronectin-coated culture plates K562-cells preloaded with Calcein were incubated in the presence of function-blocking (IIA1) or non-blocking (VC5) anti α5β1 integrin mouse monoclonal antibodies. Integrin-independent background binding of K562-cells to fibronectin was determined using 10 mM EDTA. The overall background of the assay was determined on BSA-blocked wells which do not support the adhesion of K562 cells to the surface of culture plates. Adherent cells (after washing) were lysed and fluorescence was determined.

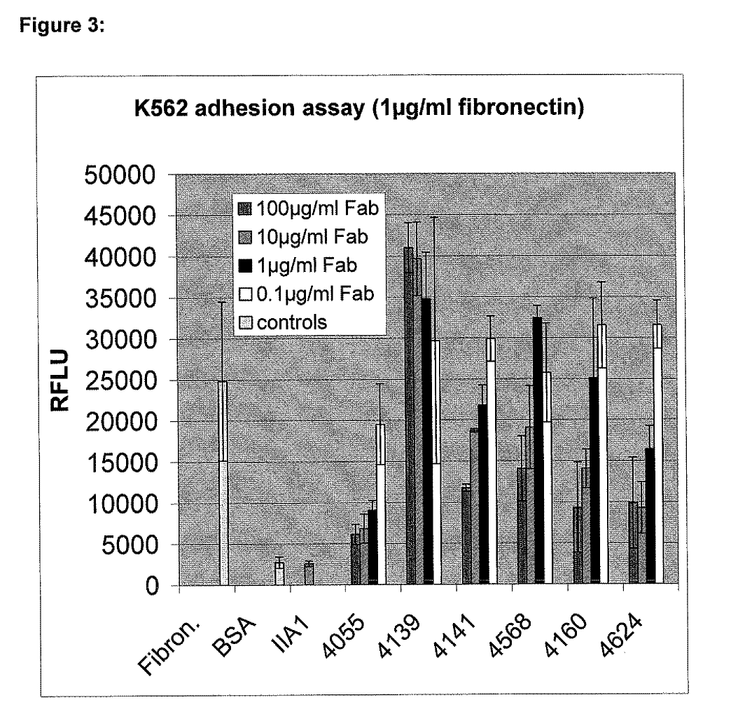

FIG. 3: Fab-mediated dose-dependent inhibition of K562 cells to fibronectin

Anti human α5β1-specific Fab were tested for their ability to inhibit the binding of fluorescent dye-loaded K562-cells to immobilized fibronectin. After adhesion, cells were lysed and fluorescence was determined as a measure for adherent cells. Fibronectin alone indicates the maximum adhesion whereas the overall background of the assay was determined on BSA-coated cells.

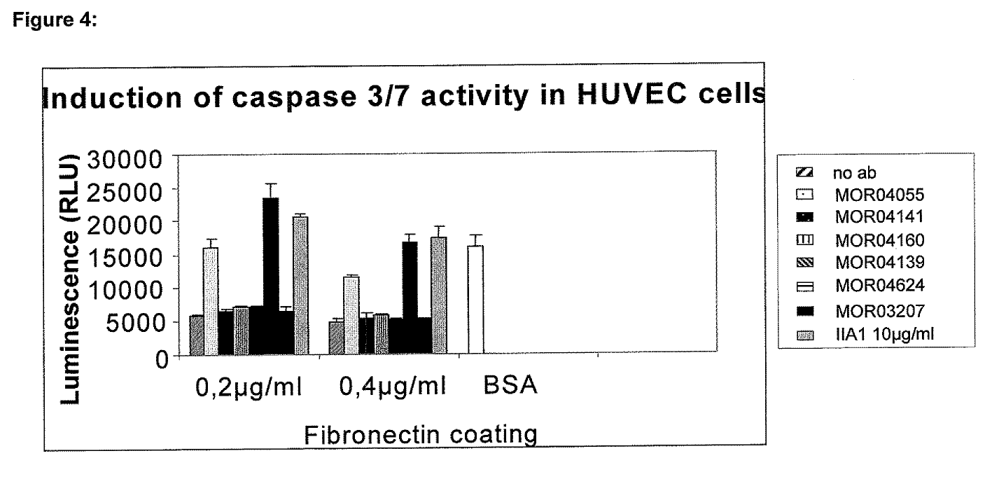

FIG. 4: Anti α5β1-function blocking antibodies induce apoptosis in endothelial cells

The induction of caspase 3/7 activation of the purified Fab in the monovalent format was determined using HUVEC cells in serum free endothelial cell medium. Caspase activity was determined using a commercially available chemoluminescent assay system (Caspase Glo, PROMEGA) according to the manufacturers' instructions.

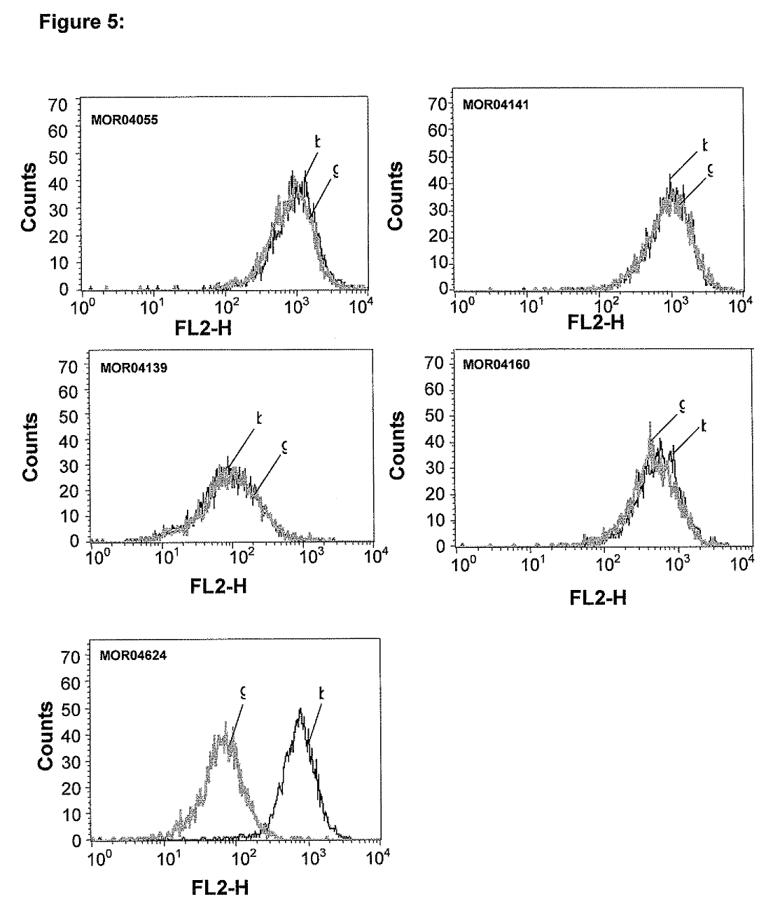

FIG. 5: Competition FACS of Fab and IIA1

The FACS-competition indicates that MOR04624 competes with the epitope of the reference antibody IIA1 on HT29α5 cells. It can be concluded that both antibodies share the similar epitope, whereas all other Fab react with unrelated binding sites on the α5β1 integrin. (black line (b)—Fab binding, green line (g)—Fab binding when competed with reference antibody IIA1).

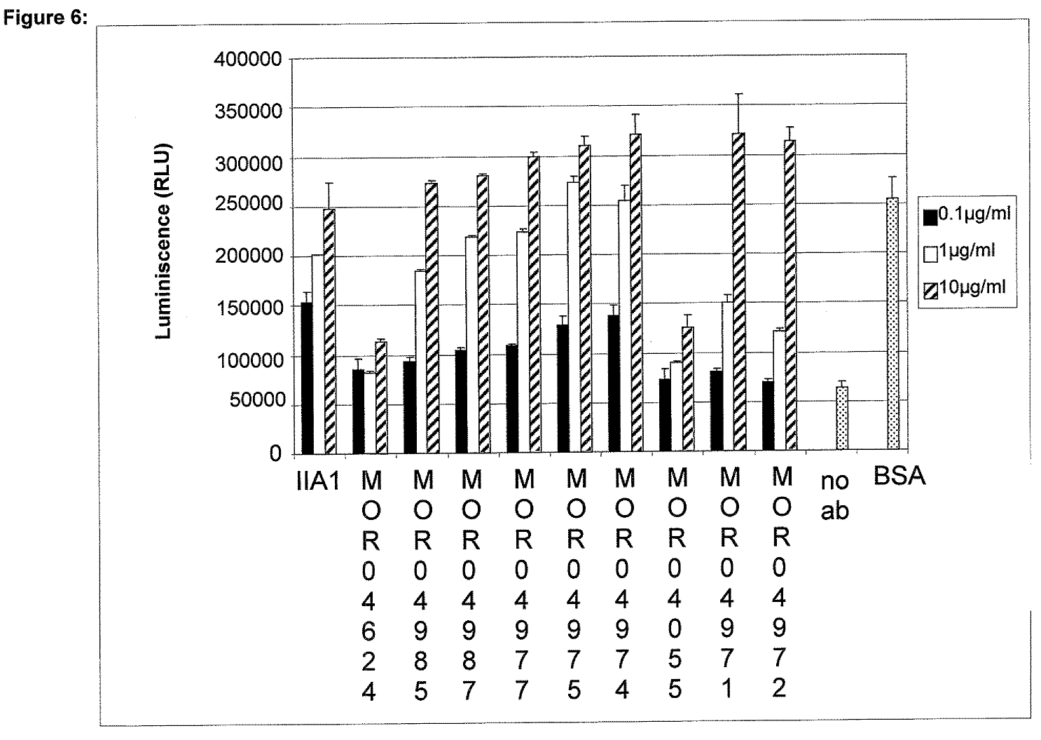

FIG. 6: Affinity-matured anti α5β1-function blocking antibodies potently induce apoptosis in endothelial cells

The induction of caspase 3/7 activation of the purified Fab in the monovalent format was determined using HUVEC cells in serum free endothelial cell medium. Caspase activity was determined using a commercially available chemoluminescent assay system (Caspase Glo, PROMEGA) according to the manufacturers' instructions.

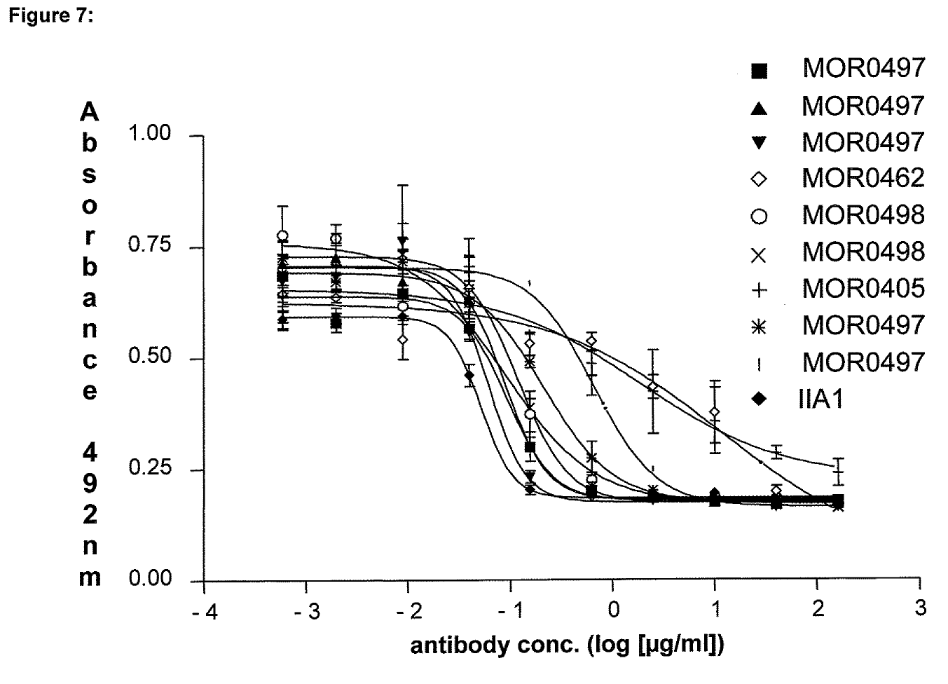

FIG. 7: Affinity-matured anti α5β1-function blocking Fab antibodies inhibit the proliferation of endothelial cells

Adherent HUVEC cells in serum free endothelial cell medium were incubated for 48 hours in presence of the indicated amount of purified Fab or reference antibody IIA1. Proliferating cells were determined with a commercially available XTT-assay according to the manufacturers' instructions. The IC50-values were determined and summarized in Table 4.

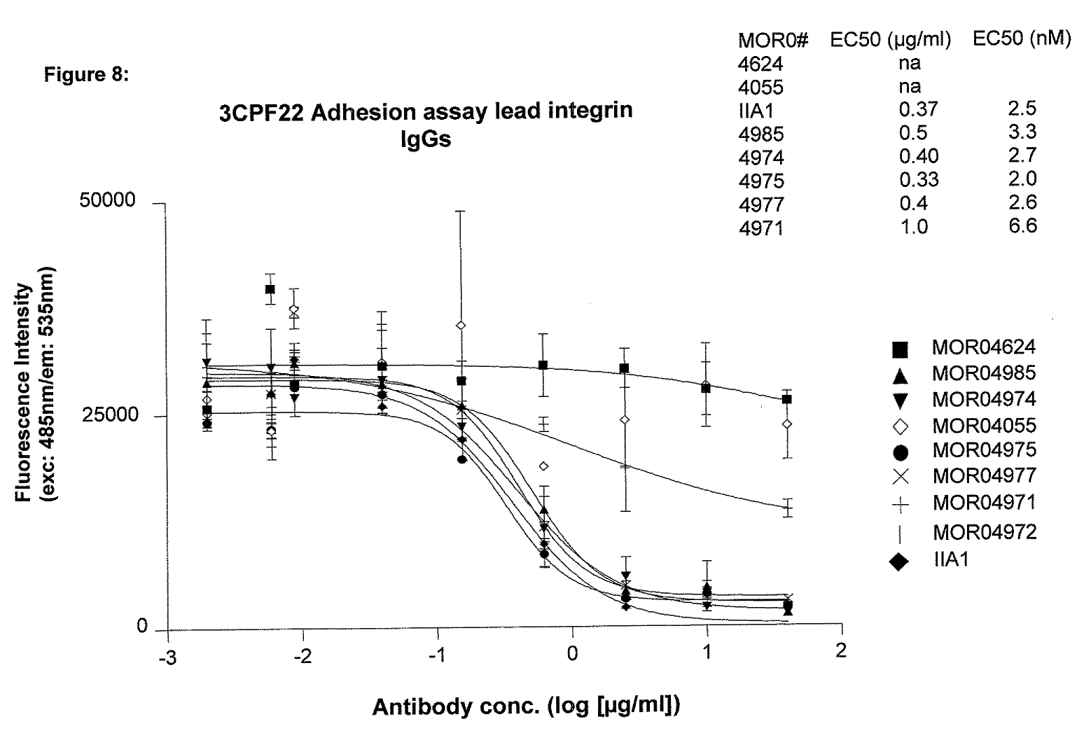

FIG. 8: Optimized IgGs in HUVEC adhesion assay

Inhibition of adhesion of HUVEC cells to fibronectin by α5β1 function blocking IgG antibodies. IgG MOR04974, MOR04975, MOR04977, MOR04985 block adhesion with a similar IC50 than IIA1. Conversion from Fab to IgG resulted in an approximately 2-fold improvement.

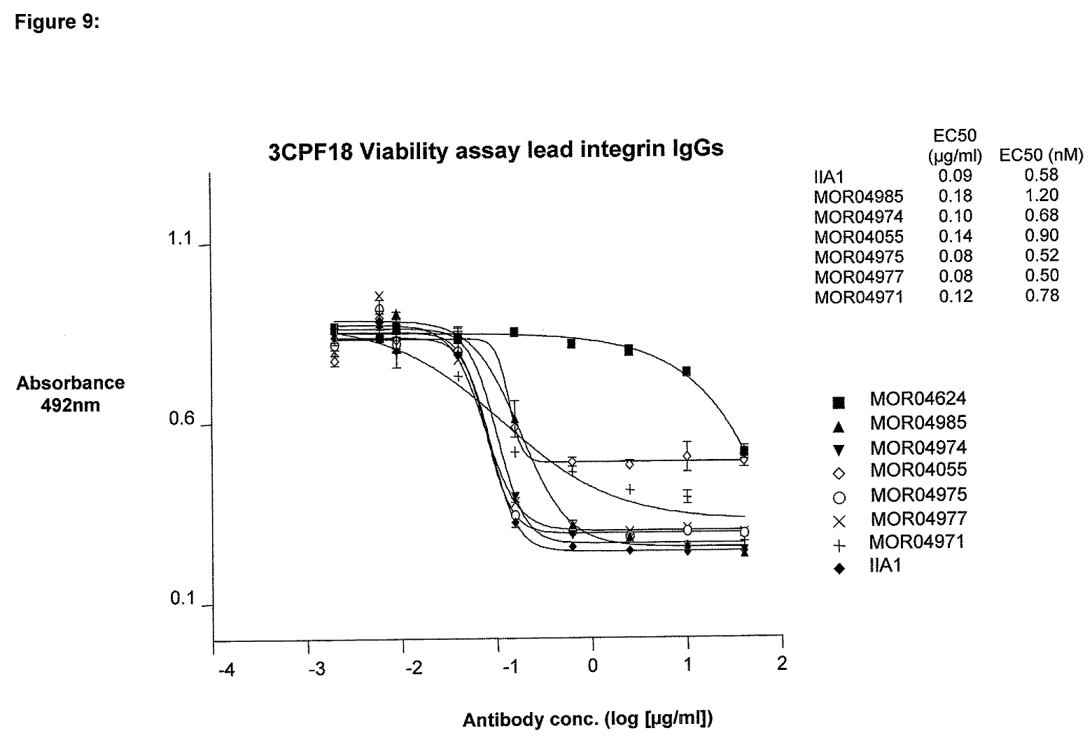

FIG. 9: HUVEC viability assay-analysis of anti-α5β1 integrin IgGs

Inhibition of viability of HUVEC cells by α5β1 function blocking IgG antibodies, HUVEC cells were plated on fibronectin coated plates, incubated with increasing concentration of IgG antibodies and survival measured after 48 h. IgG MOR04974, MOR04975, MOR04977, MOR04985 block adhesion with a similar IC50 than IIA1. Conversion from Fab to IgG resulted in an approximately 2-fold improvement.

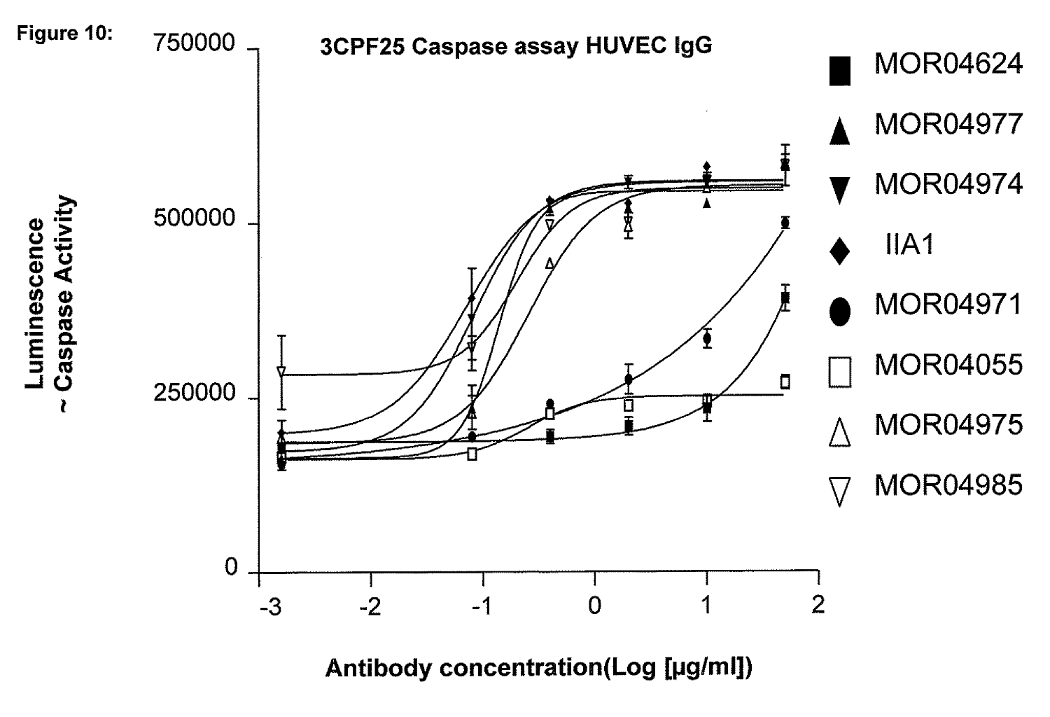

FIG. 10: HUVEC Caspase assay of anti-α5β1 integrin IgGs

The induction of Caspase 3/7 activation by the α5β1 function blocking IgG antibodies was determined using HUVEC cells in serum free endothelial cell medium. Caspase activity was determined using a commercially available chemoluminescent assay system (Caspase glo, PROMEGA) according to the manufacturers' instructions. MOR04974, MOR04975, MOR04977 and MOR04985 are similar active as the reference antibody IIA1.

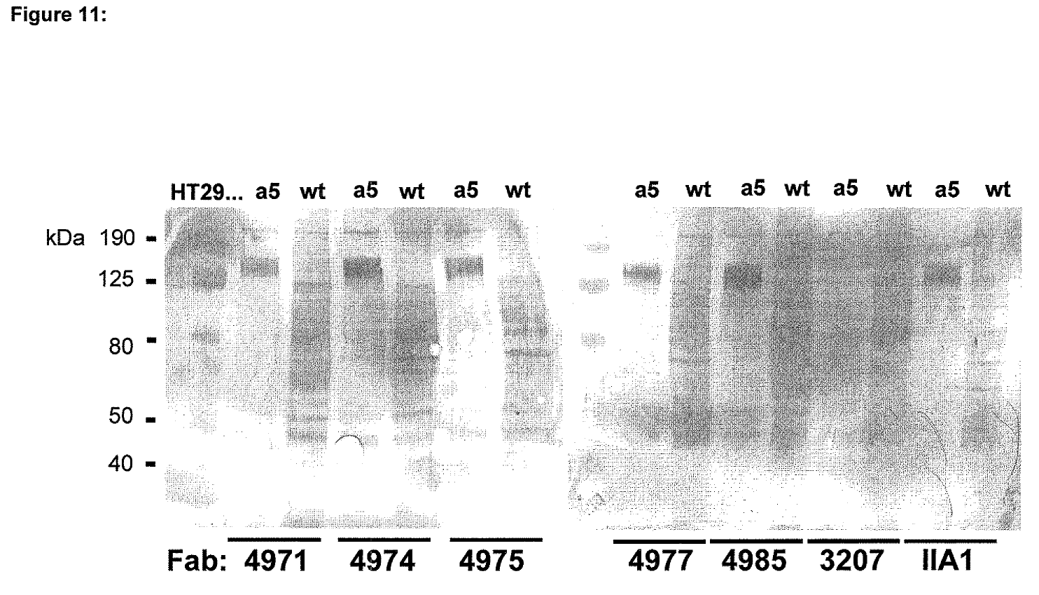

FIG. 11: Affinity-matured Fab specifically precipitate the α5β1 integrin from surface biotinylated cell lysates

Surface-biotinylated NP40-lysates of HT29α5 and HT29 wt were incubated with Fab coupled to magnetic Dyna beads. The immunoprecipitates were transferred to PVDF-membranes and analysed with streptavidin alkaline phosphatase (AP). All Fab specifically precipitated a protein of the expected size comparable to the reference antibody IIA1 out of the HT29α5 lysate whereas no protein was detectable in the HT29 wt lysate. The irrelevant Fab MOR03207 did not specifically precipitate any protein.

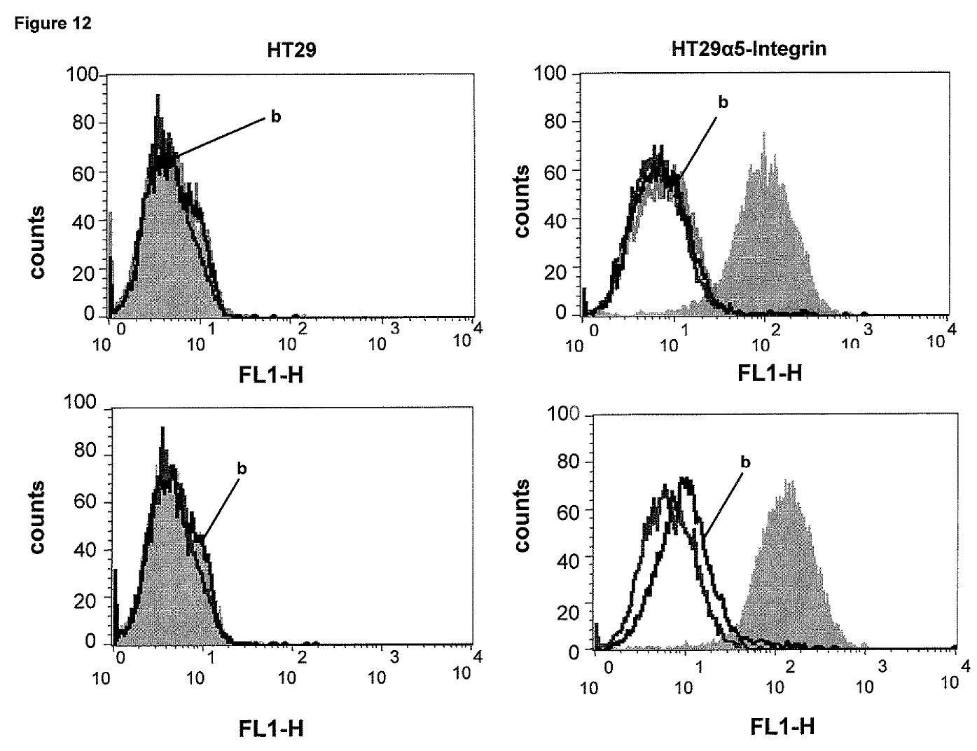

FIG. 12: Binding specificity of the anti-α5β1 integrin IgG (example MOR04974) to HT29-wt and HT29α5 (FACS measurement)

Affinity matured IgG antibodies were incubated at 10 μg/mL with 5×10⁵ HT29 wt and HT29α5 cells. Specifically bound antibodies were detected with a Cy3-labelled secondary antibody. Upper panel: IIA1 incubated with HT29 wt (left) or HT29α5-cells (right), lower panel: IgG1 MOR04974. The fluorescence shift indicates specific binding to α5 integrin and was found for MOR04975, MOR04977, MOR04985 and MOR04624 (grey area). Antibody isotype controls are negative (black lines (b)). Our anti-integrin antibodies bind to α5-chain transfected cells with the same specificity as the reference antibody IIA1.

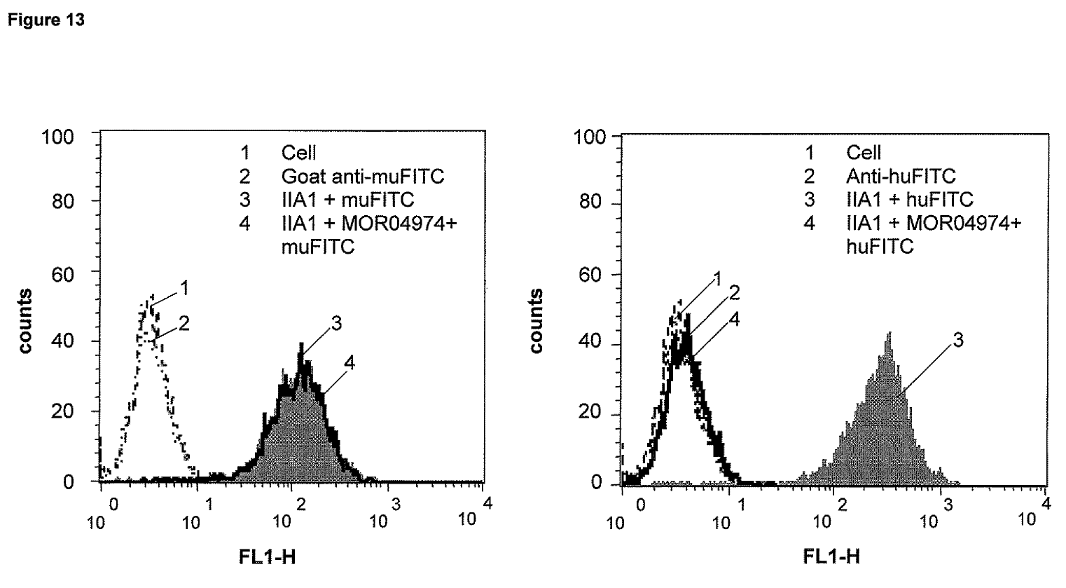

FIG. 13: Competition binding of the anti-α5β1 integrin IgG (example MOR04974) on HT29α5 cells with IIA1 (FACS measurement). The anti-α5β1 integrin IgG competes with IIA1 for an overlapping epitope.

In-house produced anti-α5β1 integrin antibodies were incubated at 1 μg/mL with 5×10⁵ HT29α5 cells which were either preincubated with 20 μg/mL IIA1 or not. Presence of IIA1 binding is demonstrated by detection with goat-anti-mouse-FITC (left panel). Binding and competition of the human antibody (MOR04974) is shown by detection with the goat-anti-human-FITC secondary antibody (right panel). This example shows the competition for MOR04974. Same result was found for MOR04975, MOR04977, MOR04985, MOR04624.

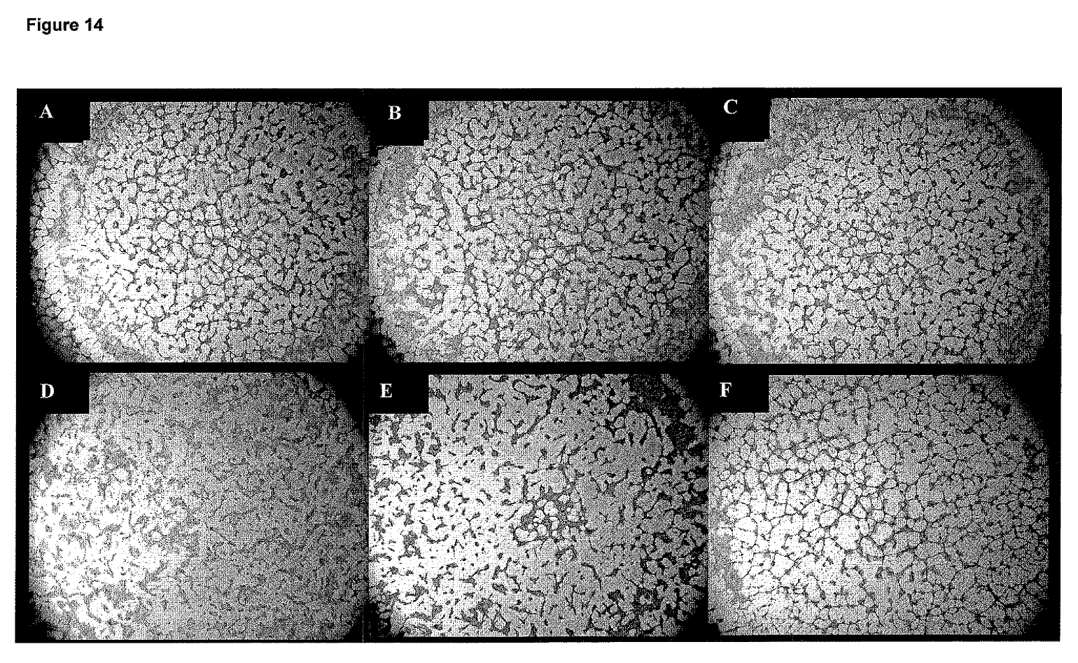

FIG. 14: Analysis of the IgG1 anti-α5β1 integrin antibodies in the tube formation assay (Example MOR04974). Affinity optimized anti-α5β1 integrin IgG1 antibodies block tube formation as efficiently as IIA1.

Early passage human endothelial vein umbilical cells (HUVECs #2519) were harvested at 60-80% confluency and 2×10⁴ cells were inoculated on Matrigel (Becton Dickinson #354234) containing wells in EBM-2 medium (Clonetics #CC3156). Antibodies were added 15 min later and tube formation was allowed to proceed for 18-24 h at 37° C. Then cells were fixed (4% formalin), permeabilized, blocked and stained with anti-CD31. Antibodies were applied at 6 nM, 3 nM, 600 pM, 300 pM, and 60 pM. Representative images are shown for the effect at 300 pM A: non-treated sample, B; human IgG1 anti-lysozyme MOR03207, C: IgG1 MOR04624, D: IgG1 MOR04974, E: IIA1, F: murine IgG1. The same result was also found for MOR04975 and MOR04977

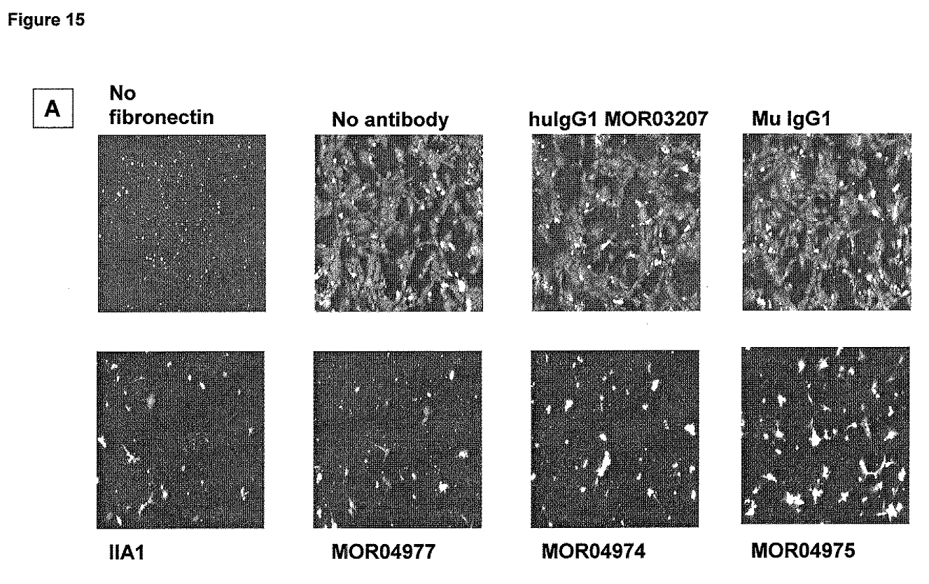

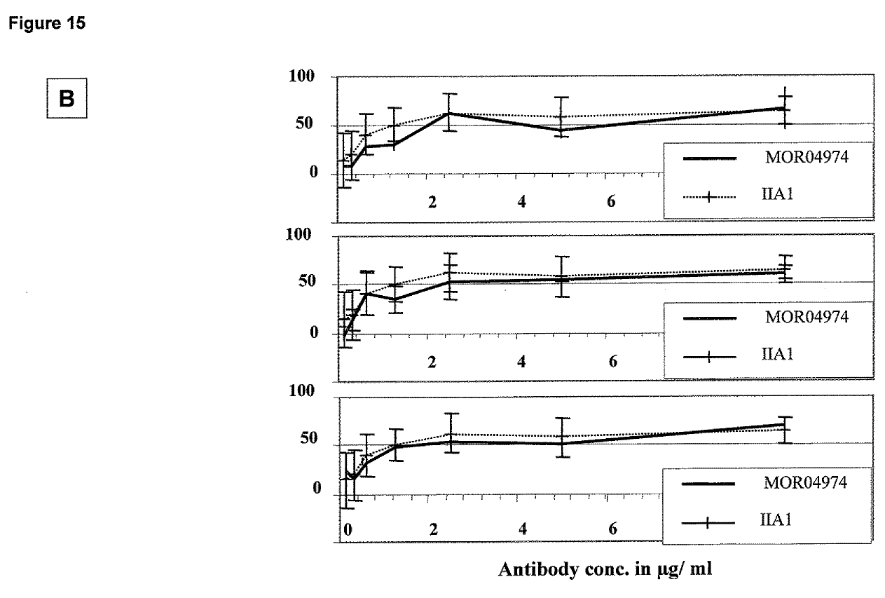

FIG. 15: Activity of the affinity optimized anti-α5β1 integrin IgG antibodies in the transwell migration assay.

The migration assay is performed in a 96-well transwell migration microplate (8 μm pores, #351163 Falcon/BD), with fibronectin as the only stimulus. The underside of fluoroblok membrane was coated with 2 μg/mL of fibronectin for 1 h at 37° C. and blocked with 2% BSA for 30 min at 37° C. Human endothelial serum-free medium (Invitrogen) containing 0.1% BSA was used as migration buffer in the upper and lower chamber. Anti-□5□1 integrin antibodies (0.6-10 μg/mL) were added to the upper chamber of each well, early passage HUVEC (2×10⁴) were added and migration of cells was allowed to proceed for 4 h at 37° C. Migrated cells on the underside of the membranes were then calcein-stained and the resulting fluorescence was determined with a Perkin Elmer1220 Victor counter at 485 nm excitation and 535 nm emission.

A: Shown images were obtained at 10 μg/mL antibody concentration. MOR04974, MOR04975, MOR04977 inhibited the migration of HUVEC as efficient as IIA1.
B: Dose-response of anti-migratory activity of MOR04974, -75, -77 (IgG4-Pro antibody isotype). IC50s (MOR04974:1 μg/ml, MOR04975: 1.5 μg/ml, MOR04977: 1 μg/ml, IIA1: 2 μg/ml)

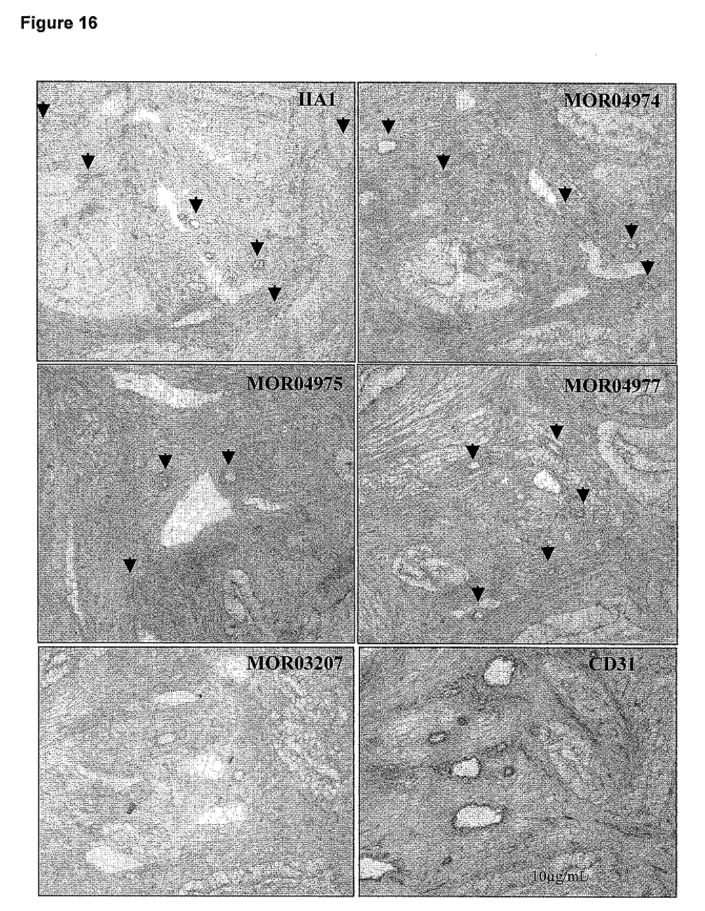

FIG. 16: IHC staining pattern of affinity-optimized IgG1 anti-α5β1-integrin antibodies on colon carcinoma tissue.

Magnification 10×, Biotinylated antibodies were titrated on serial tissue sections of colon carcinoma. Detection was done with streptavidin-alkaline phosphatase. As an example the immunohistochemical sections obtained with a concentration of 2.5 μg/mL is shown. For IIA1 and MOR04974, staining of small to intermediate size vessels and stromal compartment was found. Black arrows show the same vessels stained by both antibodies. A similar staining pattern was found for MOR04975 and MOR04977. Blue arrows indicate stained vessels. It can be concluded that the optimized anti-α5β1 integrin antibodies show staining patterns comparable to IIA1.

Tumor Targeting of the Affinity Optimized anti-α5β1 Integrin Antibodies (IgG4-Pro).

Anti-α5β1 integrin antibodies were radiolabeled with Iodine-125 (1 min, Iodogen method). Remaining immunoreactivity was determined to be 75-80% and 3 μg labelled antibody were injected into HT29α5 xenografted nude mice.

A: Tumor uptake of IgG1 MOR04974, MOR04975 and controls (reference antibody IIA1 and anti-lysozyme MOR03207), B: Tumor uptake of IgG1 MOR04977 and controls:

Antibody uptake of the anti-α5β1 integrin antibodies was as similar as for IIA1 and significantly higher compared to the irrelevant IgG1 MOR03207. We conclude from this result that the anti-α5β1 integrin antibodies specifically target the α5β1 integrin-positive HT29α5 xenografts.

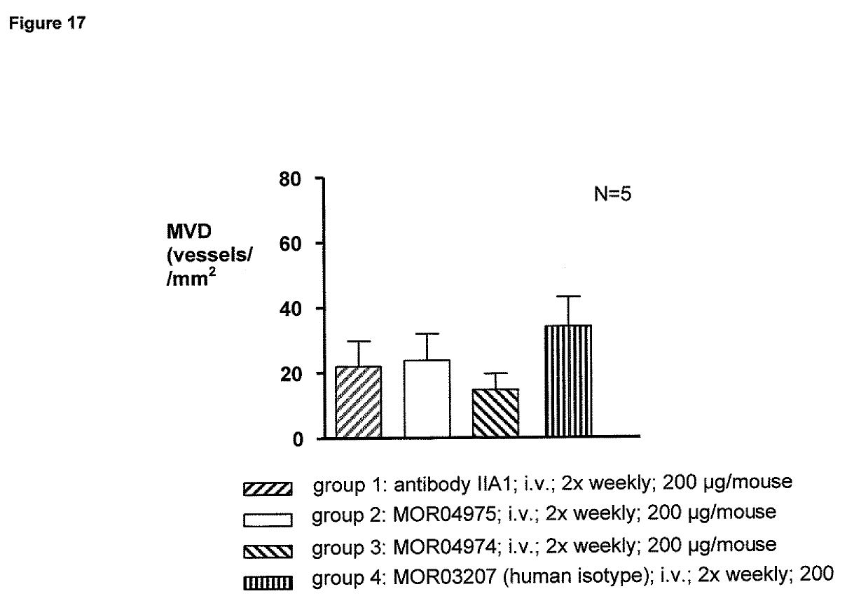

FIG. 17: Analysis of the optimized anti-α5β1 integrin IgG antibodies in the 3D in vivo spheroid surrogate model of angiogenesis.

Matrigel plugs containing spheroids of defined endothelial cell number together with VEGF and FGF2 were implanted subcutaneously into SCID mice. EC-Sprouting and vessel formation of a complex network with the mouse vasculature was analyzed after treatment with the optimized human anti-α5β1 integrin antibodies and control antibodies. The human IgG MOR04974 and MOR04975 were as efficacious as IIA1.

The invention is further illustrated in the Examples. The following Examples are, however, not to be understood as a limitation.

### EXAMPLES

### 1. Generation of Function Blocking Anti-α5β1 Integrin Antibodies

### 1.1 Screening Strategies

The mouse monoclonal antibody IIA1 binds to a conformational epitope of α5β1 integrin which is only present on activated living (endothelial) cells. To cover both selectivity and functional activity, a screening path composed of alternate pannings on isolated antigen and antigen-expressing cells in combination with functional cell-based screening assays was established for the identification of HuCAL® GOLD derived lead antibody candidates in the Fab format:

1. Selection of anti-α5β1 integrin binding Fab antibody fragments by phage display using the HuCAL®-Gold Library (MorphoSys). Panning experiments were performed on isolated antigen and antigen-expressing cells. Based on the amino acid sequences of the best antibody clones sublibraries were generated by randomization of either the VL-CDR3 or the VH-CDR2 using human CDR sequences and from which in further panning experiments even advanced binders were selected. Additional clones were obtained by cloning combinations of light and heavy chains containing interesting VL-CDR3 and VH-CDR2 in one antibody molecule (“X-cloning”).
2. Screening of enriched Fab-antibodies was done as the following. Binders of all pannings were tested for ELISA-binding on α5β1 integrin-positive and α5β1 integrin-negative cells. ELISA positive clones were then further analysed for cell binding in FACS experiments on α5-overexpressing cells and α5-negative cells. Suitable clones were then analysed in functional assays for i) cell adhesion to fibronectin ii) induction of apoptosis of HUVEC (human umbilical vein endothelial cells) and/or HDMVEC (human dermal vascular endothelial cells) iii) Affinity measurement and FACS competition assay with reference antibody IIA1 and iv) species crossreactivity.

### 1.2 Tool Generation and Assay Development

α5-Integrin Chain cDNA

The cDNA for the human α5-chain was purchased from RZPD (IMAGE-ID 6821577) and cloned into the pcDNA3-expression vector (INVITROGEN) according to standard methods.

### Purified Integrin Receptors

Detergent-solubilized human integrin receptors α5β1 (Chemicon CC1052) and α3β1 (Chemicon CC1092) were purchased from CHEMICON INTERNATIONAL (Temecula, Calif., USA). For solid phase phage display, ELISA and BiaCore-assays, integrin-batches with a purity of at least 90% were selected by non-denaturing SDS-PAGE.

### Cell Lines

The adhesion of the human chronic myelogenous leukemia cell line K562 (ATCC accession number: CCL-243) to fibronectin is solely mediated by the α5β1 integrin (16). This cell line was used in the fibronectin-mediated adhesion assay for initial functional screening. The presence of the α5β1-integrin was demonstrated by FACS-analysis using antibody IIA1 for detection (FIG. 1A).

A prerequisite for differential cell panning strategies is a model system, where the target of interest is overexpressed on a target-negative cell line. For this purpose, we have chosen the human colon carcinoma cell line HT29 (ATCC accession number: HTB-38) which expresses the β1-integrin chain, but not the α5-chain (FIG. 1B). The cDNA of the α5-chain was transfected into the parental HT29-cells using Lipofectamine according to the manufacturer's instruction. A stable α5-overexpressing clone was selected by FACS-screening using the mouse monoclonal antibody IIA1 for the specific labeling of surface expressed α5β1 integrin (FIG. 1C).

### Adhesion Assay

A sensitive adhesion assay for functional screening was established using the K562 cell line which only expresses the human α5β1 integrin. For this purpose, 96 well plates were coated with 1 μg/ml human fibronectin or BSA as a non-adhesive substrate to determine the overall background of the assay. Since the adhesion of integrins to ECM molecules is dependent on the presence Ca²⁺/Mg²⁺, 10 mM EDTA was used to determine the integrin-independent background binding on fibronectin. The function-blocking antibody IIA1 was used as a reference and a non-blocking anti α5β1 integrin mouse monoclonal antibody (VC5) served as the negative antibody-control. As expected, both EDTA, IIA1 (5 μg/ml) and BSA-coating inhibited the binding of K562-mediated adhesion, whereas VC5 (5 μg/ml) did not interfere in cell adhesion (FIG. 2).

### 1.3 Antibody Phage Display and Panning Strategies

Antibody phage display for the identification of fully human anti α5β1 integrin antibodies was performed with a HuCAL®-GOLD library according to the protocols described in literature (17-20). The following panning strategies were applied and run in parallel (Table 1):

TABLE 1
Overview panning approaches

| Panning | | | |
|---|---|---|---|
| subcode | 1^{st} round | 2^{nd} round | 3^{rd} round |
| 1298.1-3 | α5β1 integrin | α5β1 integrin | α5β1 integrin |
| | solid phase | solid phase | solid phase |
| 1298.4-6 | α5β1 integrin | K562 cells | α5β1 integrin |
| | solid phase | | solid phase |
| 1299.1-3 | K562 cells | α5β1 integrin | K562 cells |
| | | solid phase | |
| 1321.1-3 | α5β1 integrin | HT29α5 cells | α5β1 integrin |
| | solid phase | | solid phase |
| 1322.1-3 | HT29α5 cells | α5β1 integrin | HT29α5 cells |
| | p.a. HT29wt | solid phase | |
| 1322.4-6 | HT29α5 cells | HT29α5 cells | HT29α5 cells |
| | p.a. HT29wt | p.a. HT29wt | |
| 1324.1-3 | HDMVEC | α5β1 integrin | HDMVEC |
| | | solid phase | |
| 1369.1-2 | α5β1 integrin | HT29α5 cells | α5β1 integrin |
| | solid phase | p.a. HT29wt | solid phase |
| 1371.1-2 | HT29α5 cells | α5β1 integrin | HT29α5 cells |
| | p.a. HT29wt | solid phase | p.a. HT29wt |

p.a: post adsorption with HT29wt (to reduce non-specific cell surface binding)

### Results:

During the pannings 1298-1324, several thousand clones were screened. Despite the fact that various display-strategies were applied, one clone (MOR04055) which was selective in ELISA and FACS was repeatedly isolated. Besides MOR04055 which apparently binds to an immunodominant epitope, 4 additional clones were identified (MOR04139, 04141, 04160, 04568). To further increase the chance of selecting more diverse and specific integrin binders, 2 additional pannings (1369.1-2 and 1371.1-2) were performed. Here, 10 μg/ml MOR04055-Fab was added during phage display in order to suppress the enrichment of the dominating clone MOR04055. Despite of Fab-competition, all specific binders found throughout pannings 1369 were again MOR04055. In pannings 1371, one additional individual binder (MOR04624) was identified.

### 1.4 Functional Testing Fab-Antibodies

### Adhesion Assay

Antibodies obtained from the first panning approach were ranked according to their function-blocking potency in a pre-screening experiment as follows: MOR04624>MOR04055>MOR04141=MOR04568=MOR04160. MOR04139 was slightly inhibitory but did not reach 50% inhibition. The dose dependent re-testing of the antibodies at different concentrations in the K562 adhesion assay confirmed the result of our pre-screening experiment with one exception: MOR04139 did not show any dose-dependent inhibition. This antibody was not further investigated (FIG. 3).

### Induction of Apoptosis

Antibodies obtained from the first panning approach were further assessed for the apoptosis-inducing properties. Therefore 96 well plates were coated with 0.2 and 0.4 μg/ml of fibronectin for 1 hour at 37° C. and blocked with 2% BSA. 1×10⁴ HUVEC cells were incubated together with the respective antibody in serum free medium for endothelial cell culture (Gibco). After 18 hours, a caspase3/7 assay kit was used for cell lysis and quantification of caspase activity according to the procedure described by the manufacturer (Caspase Glo 3/7; Promega). At a concentration of 100 μg/ml the monovalent Fab MOR04055 and 04624 induced caspase 3/7 activity in HUVEC cells as strongly as the bivalent reference IgG IIA1 at 10 μg/ml (FIG. 4). All other Fab were negative in this assay.

### Affinity Measurements by FACS Titration

To analyse binding potency on native α5β1-integrin all antibodies were tested on α5β1-positive HUVEC cells by FACS titration (Table 2). MOR04055 had the highest binding affinity (0.9 nM) and showed an increase in the dimeric IgG format. For MOR04624 a KD in the low nanomolar range for the monovalent Fab, and an increase in KD for the dimeric IgG was found.

TABLE 2
Result of affinity determination of monovalent
Fabs and IgGs by FACS titration

| | KD (nM) | KD |
|---|---|---|
| MOR | Monovalent Fab | IgG |
| 04055 | 0.9 | 0.5 |
| 04139 | No rel. Fit | n.d. |
| 04624 | 7.5 | 3.1 |
| IgG IIA1 | — | 0.6/1.0 |

### Competition FACS of Fab and IIA1

To investigate whether the Fab antibodies share the same epitope with IIA1 or not, HT29α5 cells were incubated either with 0.5 μg/ml Fab alone or together with 10 μg/ml IIA1. Human Fabs binding to the cells were detected with goat anti human Fab-specific-PE conjugate for FACS analyses. FIG. 7 shows an overlay of Fab staining only (black lines) and Fab+IIA1 (green lines). As a result, the addition of IIA1 lead to a clear decrease in staining intensity by MOR04624. All other Fabs were not affected by IIA1. This result indicates that IIA1 and MOR04624 compete with each other for the binding to an identical or overlapping epitope, while the other 4 Fabs bind to unrelated epitopes.

### 1.5 Affinity-Maturation: Analysis of Fab and IgG Antibodies

The Fabs MOR04055 and 04624 were subjected to one round of affinity maturation. Therefore sublibaries were constructed from the parental Fab by either randomization of VL-CDR3 or VH-CDR2 (17) and subjected to phage display selections on purified α5β1 and HT29α5 cells. Positive binders of this screening were further analyzed in adhesion assay on HT29α5 cells and ranked according to their inhibitory activity. Best inhibitory potential was found for derivatives of MOR04624. Derivatives of MOR04055, MOR04568, MOR04141 did show only moderate or no significant improvement in inhibition. Based on the light and heavy chains of these clones 12 new combinations of VL-CDR3 and VH-CDR2 were cloned for further optimization (so called “X-cloning”). Best inhibitory clones and clones from X-cloning were expressed and purified and compared in vitro so that eventually 7 consolidated unique binders with improved function blocking activities were identified for further in-depth analysis (MOR04971, -72, -74, -75, -77, -85, -87).

### Induction of Apoptosis

Apoptosis-induction on HUVEC cells in vitro was measured by caspase activity and cell survival (FIG. 6 and FIG. 7). In both assays the efficacy of the monovalent Fab MOR04974, 04975 and 04977 were comparable to the bivalent mouse monoclonal reference antibody IIA1.

TABLE 3
IC50 values of Fab antibodies in the XTT-proliferation assay

| | IC₅₀ | IC₅₀ |
|---|---|---|
| | (μg/ml) | (nM) |
| IIA1 | 0.05 | 0.3 |
| MOR04624 | 11.25 | 225.0 |
| MOR04985 | 0.09 | 1.8 |
| MOR04987 | 0.12 | 2.4 |
| MOR04977 | 0.09 | 1.9 |
| MQR04975 | 0.09 | 1.8 |
| MOR04974 | 0.06 | 1.2 |
| MOR04055 | 1.87 | 37.3 |
| MOR04971 | 0.21 | 4.3 |
| MOR04972 | 0.67 | 13.3 |

In comparison to the parental Fab, the affinity matured antibodies were significantly improved (up to a factor of 190). The inhibition of proliferation of the monovalent Fab was 4-fold less efficacious than the bivalent reference antibody IIA1.

### Immunoprecipitation

To demonstrate the specificity of the Fab antibodies, NP-40 lysates of surface-biotinylated HT29α5 and HT29 wt cells were incubated with Fab coupled to magnetic Dyna-beads. IIA1 was used as a reference antibody. After intensive washing the precipitates were boiled in SDS-PAGE sample buffer under reducing conditions, blotted to PVDF-membranes and probed with streptavidine-AP. All anti-α5β1 integrin antibodies specifically precipitated a protein double kband of −135 kDa which corresponds to the expected molecular weight of the integrin chains α5 and β1 (FIG. 11) and was not found in the HT29 wt cell lysate. The same double band was found with IIA1. The irrelevant Fab MOR03207 was used as a negative control and did not precipitate this double band. This result demonstrates the high specificity of the Fab antibodies.

### Optimized IgGs in HUVEC Adhesion Assay

To investigate whether the in vitro potency of the above described Fab antibodies is further improved in the dimeric format the antibodies were converted into full IgG1 molecules according to standard technologies using the MorphoSys HuCAL IgG Vector Kit (MorphoSys AG, Munich; Germany) and were analyzed by HUVEC adhesion assay (FIG. 8), HUVEC viability assay (FIG. 9) and HUVEC apoptosis assay (FIG. 10) in comparison to the reference antibody IIA1.

Most importantly, IIA1 was included in every experiment as reference point. In this respect IgG conversion of MOR04974, -75 and -77 resulted in HuCAL IgGs with a very similar IC50 to IIA1, indicating that conversion indeed did lead to an ˜2fold improvement compared to the monovalent Fab format.

### Optimized IgGs in HUVEC Viability Assay

It was observed that after IgG conversion five binders had ˜2fold improved IC50 values compared to the Fab format. MOR04974, -75, and -77 showed a very similar efficacy in reducing HUVEC viability as reference IgG IIA1.

### Optimized IgGs in HUVEC Apoptosis Assay

From analysis of lead IgGs in the Caspase3,7 assay it could be concluded that MOR04974, -75 and -77 induced apoptosis comparably well as the reference antibody IIA1.

### 1.6 In Depth-Analysis of Affinity Optimized Anti-Integrin IgG Antibodies

### Specificity of the Affinity Optimized Anti-Integrin Antibodies

Affinity matured antibodies of the IgG-format were tested for their binding specificity by FACS analysis on HT29 wt vs. HT29α5 cells. HT29 wt cells are α5-negative but do contain the β1-integrin chain. HT29α5 but not the HT29 wt cells are specifically recognized by the IgG1-anti-integrin antibodies and the reference antibody IIA1 as indicated by the fluorescence shift. (FIG. 12). An unspecific antibody isotype control does not bind to the cells and no shift in measured fluorescence was observed. These experiments show that the lead candidate antibodies specifically recognize the α5 integrin and bind with the same specificity as the reference antibody IIA1.

Epitope Specificity of Anti-α5β1-Integrin Antibodies is Retained after Affinity Maturation and Recloning into the IgG Format

We have shown by FACS competition experiments that the Fab antibody MOR04624 and its derivatives compete with the reference antibody IIA1 for the binding to an overlapping epitope. After conversion to the IgG1 format, the anti-α5β1 integrin antibodies were tested again for binding competition with the IIA1. Binding of the IgG1 anti-α5β1 integrin antibodies MOR04974, -75, -77, -85 and MOR04624 to HT29α5 cells resulted in a shift of fluorescence which was completely inhibited when cells were preincubated with IIA1. This result confirmed the epitope competition of IIA1 and the IgG1 anti-α5β1 integrin antibodies (FIG. 13).

### Qualitative Analysis of the Anti-α5β1 Integrin IgG1 Antibodies in the Tube Formation Angiogenesis Assay.

Blockade of newly formed vessels from activated endothelial cells is considered to be one of the key inhibitory activities of the anti-α5β1 integrin antibodies. For full characterization we analyzed the affinity-optimized IgG1 anti-α5β1 Integrin antibodies in comparison with the reference antibody IIA1 in a HUVEC tube formation assay.

In this assay, 2×10⁴ human endothelial vein umbilical cells (HUVECs #2519, Promocell) were seeded on growth factor rich Matrigel (Becton Dickinson #354234) in EBM-2 medium (Clonetics #CC3156). Antibodies (6 nM, 3 nM, 600 pM, 300 pM₁ 60 pM) were added 15 min later and tube formation was allowed for 18-24 h at 37° C. Cells were then fixed and stained with anti-CD31 for photo documentation of the tube formation,

Visual analysis of the complex networks formed in the wells revealed tube formation blocking activity for all MOR04624-derived anti-α5β1 integrin antibodies with similar potency as the reference antibody (FIG. 14). At high concentrations, antibody blockade of tube formation was also found for the human and murine IgG1 isotype controls. At lower antibody concentrations (down to 300 pM), however, an activity window was observed where tube formation was only blocked in wells treated with specific antibody but not in untreated wells or wells treated with the antibody isotype control or the weak function-blocking antibody MOR04624.

### Analysis of the Anti-α5β1 Integrin IgG Antibodies in the Migration Assay

During the angiogenic process, activated endothelial cells migrate towards an angiogenic stimulus on an angiogenesis-specific provisional matrix consisting mainly of fibronectin (FN). We analyzed the optimized anti-α5β1 integrin IgG antibodies in the transwell migration assay and found blocking activity of α5β1-fibronectin dependent HUVEC migration for all anti-α5β1 antibodies with an efficacy in the same order of magnitude (1-10 μg/ml) as IIA1 (FIG. 15).

Reactivity of anti-α5β1 Integrin Antibodies

### Reactivity on Tumor and Endothelial Cell Lines

Reactivity of the anti-oα5β1 integrin antibodies was tested on various endothelial and tumor cell lines by FACS binding experiments (table 4). Binding to all tested endothelial and tumor cell lines except for HT29 wt cells, which are known to be α5-chain negative, were observed. In comparison to the reference antibody IIA1, the lead candidate antibodies bound equally well to all tested cell lines and the resulting shift in fluorescence was similar for all antibodies. Isotype control antibodies did not bind. In summary, the anti-α5β1 integrin antibodies show reactivity equivalent to IIA1 in FACS cell binding experiments.

TABLE 4
FACS reactivity of anti-α5β1 integrin antibodies on various cell lines.

| | | | | anti-α5β1* | | | | | | |
|---|---|---|---|---|---|---|---|---|---|---|
| | | anti- | anti- | | | MOR- | MOR- | MOR- | MOR- | MOR- |
| Cell line | Tissue | αVβ3 | αVβ5 | VC5 | IIA1 | 04974 | 04975 | 04977 | 04985 | 04624 |
| HDMVEC | human dermal microvascular | + | − | ++ | ++ | +++ | +++ | +++ | +++ | +++ |
| | endothelial cells | | | | | | | | | |
| HMEC | Human mammary epithel | | | | +++ | +++ | | | | |
| HPAEC | human pulmonary artery | | | | +++ | +++ | | | | |
| | endothelium | | | | | | | | | |
| HUVEC | human umbilical vein endothelial | ++ | + | +++ | +++ | +++ | | | | |
| | cells | | | | | | | | | |
| MS-1 | Mouse pancreas endothelium | | | | − | − | | | | |
| PAEC | pig aortic endothelial cells | − | +? | +++ | − | − | | | | |
| hTumorend | Human tumor endothelium | | | | ++ | +++ | +++ | +++ | +++ | +++ |
| A375 | malignant melanoma | | | | + | ++ | ++ | ++ | +/− | + |
| A549 | Human lung carcinoma | | | | ++ | +++ | +++ | +++ | +++ | ++ |
| Colo205 | Human adenocarcinoma | − | − | ++ | ++ | ++ | ++ | ++ | ++ | ++ |
| DU145 | Human prostate carcinoma | | | | +++ | +++ | +++ | +++ | +++ | +++ |
| HCT 116 | colorectal carcinoma | | | | + | ++ | ++ | ++ | +/− | + |
| HT-1080 | Fibrosarcoma | | | | ++ | +++ | +++ | +++ | + | +++ |
| MCF-7 | Human breast carcinoma | +/− | ++ | + | + | + | + | + | + | + |
| MDA-MB- | Human mammary carcinoma | ++ | ++ | +++ | ++/+++ | ++ | ++ | ++ | + | ++ |
| 231 | | | | | | | | | | |
| PC-3 | human prostata adenocarcinoma | + | + | ++ | ++ | | | | | |
| U251 | Human glioblastoma | + | + | ++ | ++ | ++ | ++ | ++ | ++ | ++ |
| HT29 | Human adenocarcinoma | +/− | + | − | − | − | − | − | − | − |
| HT29α5 | HT29 transf. with α5 chain | | | | +++ | +++ | +++ | +++ | +++ | +++ |

+ = weak shift,
++ strong shift (1 log fluorescence),
+++ very strong shift (2 log Fluorescence);
*mIgG1 and huIgG1 as antibody controls were used as standard controls and binding was negative; Antibody material: anti-human CD51/61 chemicon #CBL 544, anti-αvβ5 chemicon #Mab 20192, non function-blocking α5β1 integrin antibody VC5 Pharmingen #555650, anti-α5β1 integrin antibody IIA1 Pharmingen #55561

Reactivity of anti-α5β1 Antibodies on Normal and Tumor Tissue Sections—Immunohistochemistry

The affinity-optimized anti-α5β1 integrin antibodies were analyzed in immunohistochemistry experiments on different tissue section and the specific reactivity profile of the anti-integrin antibodies on the respective tissues very much resembled the staining of IIA1. In summary, we conclude that the our anti-α5β1 integrin antibodies show staining patterns comparable to IIA1 (FIG. 16).

### In Vivo Characterization of the Affinity Optimized Anti-α5β1 Integrin IgG Antibodies

### Demonstration of In Vivo Targeting in Xenografted Nude Mice

The in vivo targeting properties of the optimized anti-5 μl integrin antibodies in comparison to IIA1 were compared in nude mice carrying xenografts of HT29α5 cells.

Radiolabeling of the optimized anti-α5β1 integrin antibodies (IgG4-Pro) was performed with iodine-125 according to the iodogen-method for 1 min according to standard procedures. Immunoreactivity was measured in a cell-binding assay (“Lindmo assay”). 50 ng of radiolabeled antibody were incubated with increasing numbers (0.25 to 10 Mio) of α5β1 integrin-positive cells for 2 h at 4° C. Then cells were washed and bound radioactivity was determined in a scintillation counter. The quotient of total counts/bound counts was plotted against 1/cell number and data were fitted with a non-linear regression model. From the intersection with the y-axis the remaining immunoreactivity at infinite antigen density was calculated and found to be 75-80% for all anti-α5β1 integrin antibodies.

The human anti-α5β1 integrin antibodies accumulated within from 24 hours to the HT29α5 xenografts with >10% I/g lasting 96 hours for all analyzed antibodies except MOR04975 which rapidly decreased after 48 hours to less than 5% ID/g after 72 h. MOR04974 reached its peak value after 48 hours with 18% ID/g and MOR04977 after 72 h with 18% ID/g. In comparison the murine IIA1 antibody accumulated within from 24 h at the HT29α5 xenografts with >10% ID/g lasting for up to 96 h. For the non-specific anti-lysozme antibody MOR03207 less than 3% ID/g were found at any time point. From these results a specific targeting of α5β1-positive HT29α5 xenografts can be concluded. The in vivo targeting of the anti-α5β1 integrin antibodies MOR04974 and MOR04977 is similar to IIA1 and at single time points even superior.

In Vivo Efficacy of anti-α5β1 Integrin Antibodies from Surrogate Animal Models of Angiogenesis

As the reference antibody IIA1, the anti-α5β1 integrin antibodies are not cross-reactive with mouse and rat α5β1 integrin. Therefore analysis of the in vivo therapeutic efficacy and demonstration of the specific in vivo antiangiogenic effect in animal models is difficult and has to be performed in surrogate models of angiogenesis.

In vivo comparison of the anti-α5β1 integrin IgG antibodies with IIA1 has been performed in the 3D in vivo spheroid surrogate model of angiogenesis (FIG. 17).

For this model spheroids of defined endothelial cell number were mixed with collagen which was allowed to polymerize in a 24 well plate. EC spheroids in matrigel plugs containing VEGF and FGF2 were then implanted subcutaneously into SCID mice where the stimulated ECs formed a complex three dimensional network of human capillaries that anastomosed with the mouse vasculature. Anti-α5β1 integrin antibodies (200 μg) were given twice weekly for three weeks. At day 21 the study was terminated, matrigel plugs were removed and examined for blood vessel density. As for the reference antibody IIA1 treatment with the optimized anti-α5β1 integrin IgG antibodies MOR04974 and MOR04975 reduced the microvessel density in the matrigel plugs by a factor of two to approximately 20 microvessels per mm2 while treatment with the irrelevant human anti-lysozyme antibody MOR03277 resulted in about 40 microvessels per mm2.

Based on this result it can be concluded that the optimized human anti-α5β1 integrin antibodies MOR04974 and MOR04975 have comparable in vivo anti-angiogenic efficacy as IIA1 in the 3D in vivo spheroid surrogate model of angiogenesis.

### CONCLUSION

In in vitro experiments the best inhibitory properties in Fab as well as IgG1 format were consistently found for MOR4974, -75, -77. All three IgGs are comparable to reference mAb IIA1. These binders are derivatives of MOR04624. In in vivo experiments the fully human and optimized IgG MOR04974, -75, -77 were demonstrated to target efficiently tumor xenografts in nude mice and in the 3D spheroid model of angiogenesis MOR04974 and MOR04975 were as efficacious as the reference antibody IIA1.

The amino acid sequences of the V chains of the
above antibodies are shown in Table 4:
Parental MOR04624

| | | |
|---|---|---|
| Final hlgG1 | | |
| kappa | VH-h-IgG1-vector | VL-h-kappa-vector |
| MOR04974 | MOR04985 | MOR04990 |
| MOR04975 | MOR04985 | MOR04991 |
| MOR04977 | MOR04987 | MOR04989 |
| MOR04985 | MOR04985 | MOR04624 |

MOR04624
VLκ (SEQ ID NO: 1)
diqmtqspsslsasvgdrvtitcrasqgissnlnwyqqkpgkapklliya
asnlqsgpsrfsgsgsgtdftltisslqpedfavyycqqysdqsytfgqg
tkveikrt
VH (SEQ ID NO: 2)
qvqlvesggglvqpggslrlscaasgftfssygmswvrqapgkglewvss
isysdsntyyadsvkgrftisrdnskntlylqmnslraedtavyycargl
gdyghhhglsgifdywgqgtlvtvss
MOR04055
VLλ3 (SEQ ID NO: 3)
dieltqppsvsvapgqtariscsgdsigeqyahwyqqkpgqapviviydd
nkrpsgiperfsgsnsgntatltisgtqaedeadyycgsytltntasvfg
ggtkltvlg
VH3 (SEQ ID NO: 4)
qvqlvesggglvqpggslrlscaasgftfsnyamnwvrqapgkglewvsr
isgsgsdtyyadsvkgrftisrdnskntlylqmnslraedtavyycareg
efgfmystlvfdswgqgtlvtvss
MOR04971
VLλ3 (SEQ ID NO: 5)
dieltqppsvsvapgqtariscsgdsigeqyahwyqqkpgqapvlviydd
nkrpsgiperfsgsnsgntatltisgtqaedeadyycssytyssdasvfg
ggtkltvlg
VH3 (SEQ ID NO: 6)
qvqlvesggglvqpggslrlscaasgftfsnyamnwvrqapgkglewvsa
ihdnghtyypdsvkgrftisrdnskntlylqmnslraedtavyycarege
fgfmystlvfdswgqgtlvtvss
MOR04974
VLκ (SEQ ID NO: 7)
diqmtqspsslsasvgdrvtitcrasqgissnlnwyqqkpgkapklliya
asnlqsgpsrfsgsgsgtdftltisslqpedfatyycqqyasprqtfgqg
tkveikrt
VH (SEQ ID NO: 8)
qvqlvesggglvqpggslrlscaasgftfssygmswvrqapgkglewvsg
irakqsgyatdyaapvkgrftisrdnskntlylqmnslraedtavyycar
glgdyghhhglsgifdywgqgtlvtvss
MOR04975
VLκ (SEQ ID NO: 9)
diqmtqspsslsasvgdrvtitcrasqgissnlnwyqqkpgkapklliya
asnlqsgpsrfsgsgsgtdftltisslqpedfatyycqqyefgiqtfgqg
tkveikrt
VH (SEQ ID NO: 10)
qvqlvesggglvqpggslrlscaasgfttssygmswvrqapgkglewvsg
irakqsgyatdyaapvkgrftisrdnskntlylqmnslraedtavyycar
glgdyghhhglsgifdywgqgtlvtvss
MOR04977
VLκ (SEQ ID NO: 11)
diqmtqspsslsasvgclrvtitcrasqgissnlnwyqqkpgkapklliy
aasnlqsgpsrtsgsgsgtdftltisslqpedfatyycqqyssnpqttgq
gtkveikrt
VH (SEQ ID NO: 12)
qvqlvesggglvqpggslrlscaasgftfssygmswvrqapgkglewvsf
iepkwrggathyaasvkgrftisrdnskntlylqmnslraedtavyycar
glgdyghhhglsgifdywgqgtlvtvss
MOR04985
VLκ (SEQ ID NO: 13)
diqmtqspsslsasvgdrvtitcrasqgissnlnwyqqkpgkapklliya
asnlqsgpsrfsgsgsgtdftltisslqpedfavyycqqysdqsytfgqg
tkveikrt
VH (SEQ ID NO: 14)
qvqlvesggglvqpggslrlscaasgftfssygmswvrqapgkglewvsg
irakqsgyatdyaapvkgrftisrdnskntlylqmnslraedtavyycar
glgdyghhhglsgifdywgqgtlvtvss

### 2. CONCLUSION

Anti-α5β1 integrin function blocking antibodies have only been available in a chimeric antibody format. Approaches for a fully humanization have failed. Application of such antibodies in the clinical setting may induce an immune response in human patients. Especially for a chronically applied anti-angiogenic compound this may lead to increased dosing or even severe side effect which may lead to the early termination of treatment.

We have identified fully human α5β1 integrin function blocking antibodies with an excellent biological profile. It is advantageous to existing murine and chimeric antibodies due to its fully human nature which will guarantee lack of side effects in clinical settings as far as possible. It is expected that the probability of inducing an immune response against this molecule with severe side effects and or increased doses is much lower. Therefore these molecules are much more suitable for the application in human medicine, e.g. for the treatment of solid tumors.

### REFERENCES

1) Carmeliet P and Jain R K (2000) Nature 407: 249-257
2) Kim S et al. (2002) J Clin Invest 110: 933-941
3) Kim S et al. (2002) Am J Pathol 156: 1345-1362
4) Kim S et al. (2002) J Biol Chem 275: 33920-33928
5) Cheresh D A and Stupack D G (2002) Nat Med 8: 193-194
6) George E L et al. (1993) Development 119: 1079-1091
7) George E L et al. (1997) Blood 90: 3073-3081
8) Yang et al. (1993) Development 119: 1093-1105
9) Goh K L et al. (1997) Development 124: 4309-4319
10) Taverna D and Hynes R O (2001) Cancer Res 61: 5255-5261
11) Francis SE et al. (2002) Artherioscler Thromb Vasc Biot 22: 927-933
12) McDonald D and Choyke P L (2003) Nat Med 9: 713-725
13) Bakre M M et al. (2002) Nat Med 8: 995-1003
14) Finck B (2004) SRI conference “Angiogenesis: New Opportunities And Solutions For Drug Development”, Cambridge, Mass.
15) Symington B E (1989) J Biol Chem 264: 13258-13266
16) Knappik A et al. (2000) J Mol Biol 296: 57-86
17) Krebs B et al. (2001) J Immunol Methods 254: 67-84
18) Rauchenberger R et al. (2003) J Biol Chem 278: 38194-38205
19) Lohning C, U.S. Pat. No. 6,753,136
20) Magnussen et al. (2005) Cancer Research 65: 2712-2721
21) Yao et al. (2006) Cancer Research 66: 2639-2649.

1. A human or humanized antibody or an antigen-binding fragment thereof which binds to α5β1 integrin with an affinity of ≦100 nM and which inhibits the adhesion of α5β1 integrin-expressing cells to its receptor in vitro and in vivo.

2. The antibody or fragment of claim 1 which binds to α5β1 integrin with an affinity of ≦10 nM.

3. The antibody or antibody fragment of claim 1 which inhibits the adhesion of the K562 cell line in vitro.

4. The antibody or antibody fragment of claim 1 which comprises:
(a) a VH-region selected from
(i) amino acid sequence SEQ ID NO: 1 (MOR04624), SEQ ID NO: 3 (MOR04055) or at least one H-CDR1, H-CDR2 and/or H-CDR3 region of one of said VH regions, or
(ii) an amino acid sequence derived from a sequence of (i) by alteration of at least one H-CDR region, and/or
(b) a VL-region selected from
(i) amino acid sequence SEQ ID NO: 2 (MOR04624), SEQ ID NO: 4 (MOR04055) or at least one L-CDR1, L-CDR2 and/or L-CDR3 region of one of said VL regions, or
(ii) an amino acid sequence derived from a sequence of (i) by alteration of at least one L-CDR region.

5. The antibody or antibody fragment of claim 4, comprising a VH-region derived from a VH-region of (a) (i) by randomization of the H-CDR2 region.

6. The antibody or antibody fragment of claim 4 comprising a VL-region derived from a VL-region of (b) (i) by randomization of the L-CDR3 region.

7. The antibody or antibody fragment of claim 4 comprising a VH- and/or VL-region derived from a VH-region of (a) (i) and/or from a VL-region of (b) (i) by shuffling of the antibody chains.

8. The antibody or antibody fragment of claim 4 comprising
(a) a VH-region selected from amino acid sequence SEQ ID NO: 5 (MOR04971), SEQ ID NO: 7 (MOR04974), SEQ ID NO: 9 (MOR04975), SEQ ID NO: 11 (MOR04977), and SEQ ID NO. 11 (MOR04985) or at least one H-CDR1, H-CDR2 and/or H-CDR3 region of said VH-regions, and/or
(b) a VL-region selected from amino acid sequence SEQ ID NO: 6 (MOR04971), SEQ ID NO: 8 (MOR04974), SEQ ID NO: 10 (MOR04975), SEQ ID NO: 12 (MOR04977) and SEQ ID NO: 14 (MOR04985), or at least one L-CDR1, L-CDR2 and/or L-CDR3 region of said VL-regions.

9. An antibody or antibody fragment comprising the VH region of SEQ ID NO: 1 and the VL region of SEQ ID NO: 2 (MOR04624) or at least one H-CDR1-, H-CDR-2, H-CDR3-, L-CDR1-, L-CDR2- or L-CDR3-region thereof.

10. An antibody or antibody fragment comprising the VH region of SEQ ID NO: 3 and the VL region of SEQ ID NO: 4 (MOR04055) or at least one H-CDR1-, H-CDR-2, H-CDR3-, L-CDR1-, L-CDR2- or L-CDR3-region thereof.

11. An antibody or antibody fragment comprising the VH region of SEQ ID NO: 5 and the VL region of SEQ ID NO: 6 (MOR04971) or at least one H-CDR1-, H-CDR-2, H-CDR3-, L-CDR1-, L-CDR2- or L-CDR3-region thereof.

12. An antibody or antibody fragment comprising the VH region of SEQ ID NO: 7 and the VL region of SEQ ID NO: 8 (MOR04974) or at least one H-CDR1-, H-CDR-2, H-CDR3-, L-CDR1-, L-CDR2- or L-CDR3-region thereof.

13. An antibody or antibody fragment comprising the VH region of SEQ ID NO: 9 and the VL region of SEQ ID NO: 10 (MOR04975) or at least one H-CDR1-, H-CDR-2, H-CDR3-, L-CDR1-, L-CDR2- or L-CDR3-region thereof.

14. An antibody or antibody fragment comprising the VH region of SEQ ID NO: 11 and the VL region of SEQ ID NO: 12 (MOR04977) or at least one H-CDR1-, H-CDR-2, H-CDR3-, L-CDR1-, L-CDR2- or L-CDR3-region thereof.

15. An antibody or antibody fragment comprising the VH region of SEQ ID NO: 13 and the VL region of SEQ ID NO: 14 (MOR04985) or at least one H-CDR1-, H-CDR-2, H-CDR3-, L-CDR1-, L-CDR2- or L-CDR3-region thereof.

16. The antibody or antibody fragment of claim 1 which is an IgG antibody, e.g. a human or humanized IgG1, IgG2, IgG3 or IgG4 antibody or a fragment thereof, e.g. a Fab, Fab′ or F(ab)₂ fragment.

17. The antibody or antibody fragment of claim 1 which is a recombinant antibody, e.g. a single-chain (sc) antibody, or a fragment thereof, e.g. a sc Fv fragment.

18. The antibody or antibody fragment of claim 1 in the form of a conjugate with a therapeutic agent.

19. The antibody or antibody fragment of claim 18 wherein the therapeutic agent is selected from radiotherapeutical agents and chemotherapeutical agents.

20. The antibody or antibody fragment of claim 19 wherein the radiothera-peutical agent is I¹²⁵, I¹³¹ or Y⁹⁰.

21. The antibody or antibody fragment of claim 1 in the form of a fusion polypeptide.

22. The antibody or antibody fragment of claim 21 as a fusion polypeptide with a cytokine or as a bispecific antibody.

23. The antibody or antibody fragment of claim 1 in the form of a conjugate with a detectable labelling group.

24. The antibody or antibody fragment of claim 23, wherein the detectable labelling group is selected from radioactive, NMR, dye, enzyme and fluorescent labelling groups.

25. The antibody or antibody fragment of claim 24, wherein the detectable radioactive labelling group is selected from I¹²⁵, I¹³¹ or Y⁹⁰.

26. A pharmaceutical composition comprising as an active agent an antibody or antibody fragment of claim 1.

27. The composition of claim 26 for the prevention or treatment of hyperproliferative disorders.

28. The composition of claim 26 for the prevention or treatment of cancer.

29. The composition of claim 26 for the prevention or treatment of colon carcinoma.

30. The composition of claim 26 for the prevention or treatment of tumors.

31. A diagnostic composition comprising as a diagnostic reagent an antibody or antibody fragment of claim 1.

32. The composition of claim 31 for the diagnosis of hyperproliferative disorders or a predisposition therefor.

33. The composition of claim 32 for the diagnosis of cancer or a predisposition therefor.

34. The composition of claim 26 for use in human medicine.

35. A nucleic acid encoding an antibody or antibody fragment of claim 1.

36. The nucleic acid of claim 35 which is operatively linked to an expression control sequence.

37. A vector or vector system which comprises a nucleic acid of claim 35.

38. A cell which is transformed with a nucleic acid of claim 35.

39. A non-human organism which is transformed with a nucleic acid of claim 35.

40. A method for preparing a polypeptide of claim 1, wherein a cell or a non-human organism, transformed with a nucleic acid encoding for said polypeptide, is cultivated under conditions, under which the polypeptide is expressed and the expressed polypeptide recovered.

41. A method for the prevention or treatment of α5β1 integrin associated disorders or a predisposition therefore comprising administering a polypeptide according to claim 1.

42. The method of claim 41 or the manufacture of a medicament for the prevention or treatment of cancer.

43. A method for the diagnosis of α5β1 integrin associated disorders or a predisposition therefore, using a polypeptide according to claim 1.

44. The method of claim 43 for the manufacture of a reagent for the diagnosis of cancer
